(19) United States
(12) Patent Application Publication
(10) Pub. No.: US 2012/0171257 A1
(43) Pub. Date: Jul. 5, 2012

(54) Cell-guiding fibroinductive and angiogenic scaffolds for periodontal tissue engineering

(76) Inventors: Bülend Inanç, Istanbul (TR); Levent Inanç, Istanbul (TR)

(21) Appl. No.: 13/392,888

(22) PCT Filed: Sep. 12, 2009
(86) PCT No.: PCT/IB09/53996

§ 371 Date: Feb. 28, 2012

(51) Int. Cl. A61K 38/18; A61P 19/00; A61P 9/00; A61K 9/00

(52) U.S. Cl. 424/400; 514/7.6; 514/9.1; 514/8.5; 514/8.6; 514/8.2; 514/8.9; 514/8.8; 514/8.1

(57) ABSTRACT

Disclosed are methods for producing cell-guiding fibroinductive and angiogenic tissue engineering scaffolds composed of biodegradable and biocompatible natural biopolymers, synthetic polymers and/or their combination, incorporating growth and differentiation factors, growth hormone and chemoattractants, with interconnected pores and channels-containing microarchitecture inducing the regenerative cell migration, adhesion, proliferation and differentiation from the healthy tissues surrounding the periodontal defects, thereby facilitating the functional periodontal tissue regeneration. The methods for the application of the cell-guiding fibroinductive and angiogenic scaffolds in the surgical treatment of periodontal tissue defects resulted from destructive periodontal diseases are also provided.

### TECHNICAL FIELD

The present invention relates to the producing of the cell-guiding fibroinductive and angiogenic scaffolds for use in tissue engineering for periodontal regeneration, joint ligaments regeneration, muscle tendon regeneration, periosteum regeneration, and the methods for their modification and use thereof.

The invention further is based on utilizing the chemotactic and proliferative effects of multiple growth factors and biomaterial scaffolds with defined architectural and topologic characteristics to guide the migration, proliferation and functional induction of progenitor cells with cementogenic, fibrogenic, osteogenic and angiogenic tissue regeneration capabilities. The invention also relates to induction of newly regenerated functional connective tissue formation in tendon and ligament tissue engineering and more particularly in periodontal tissue engineering.

### BACKGROUND ART

The alveolar bone around tooth roots, cementum on the root surfaces and the periodontal ligament providing the connection between the two mineralized tissues are all parts of a functional unit ensuring the localization and physiologic function of the teeth inside the jaw bones. Inflammatory periodontal diseases are the most common cause of periodontal destruction and are widespread in general population (Albandar J M. Dent Clin North Am 2005; 49(3):517-32). Although rapidly progressive forms like aggressive periodontitis affects younger individuals, chronic periodontitis affects adult population as incidence increases with age (Albandar J M. Dent Clin North Am 2005; 49(3):517-32, Oh et al. J Clin Periodontol 2002; 29(5):400-10). The prevention and treatment of the destruction of soft and mineralized tissues caused by inflammatory periodontal diseases and the regeneration of lost supporting structures are among the principal aims of the periodontology. Important advances during the 20^{th} century in this field allowed the development of various surgical procedures for the correction of the anatomical defects caused by the diseases. Soft and mineralized tissue loss can be restored by various grafting procedures. However, the majority of these techniques are allowing only the correction, but not the regeneration of the periodontal apparatus. The development of the guided tissue regeneration (GTR) concept in the 1980's demonstrated the regenerative potential of the periodontal structures (Gotlow et al. J Clin Periodontol. 1986; 13(6):604-16). This technique is based on the principle of exclusion of gingival fibroblastic and epithelial cells from the periodontal defect site by barrier membranes, after root planning and debridement in periodontal surgery. This in turn allows the cell types capable of regenerating the functional periodontal apparatus, the periodontal ligament fibroblastic cell progenitors residing in the neighboring healthy tissues to repopulate the area and ensure regeneration to certain extent. However, the outcome is variable and depends on multiple factors such as age, genetics, defect size and type, etc., and the amount of regeneration is often limited. The underlying mechanisms may involve the difficulty of the periodontal progenitor cells to migrate on the root surfaces affected by the disease and changes in cementum structure triggered by pathological processes (Grzesik W J, Narayanan A S. Rev Oral Biol Med. 2002; 13(6):474-84). The use of bone graft materials in bone defects provides osteoconductive scaffolds for bone reconstruction, but the functional attachment between the newly formed bone and the tooth root surface does not form. Enamel matrix proteins have been derived from the animal tooth germs in developmental stage with the aim to recapitulate the developmental processes. These enamel matrix derivatives contain developmental proteins like amelogenin and have been shown to have beneficial effects on periodontal regeneration by stimulating periodontal progenitor cells (Sculean et al. J Periodontal Res. 1999; 34(6):310-22). However, these substances per se are insufficient to promote complete regeneration. They also do not provide any defined three-dimensional extracellular matrix for the regenerative cells. Various patents aiming to develop different scaffolds for periodontal regeneration reflecting the state-of-the art are presented below:

U.S. Pat. No. 5,447,725; “Methods for aiding periodontal tissue regeneration”, (Authors: Damani N C, Mohl D C, Singer Jr. R E; Publication Date: Sep. 5, 1995). Compositions containing bioresorbable polymers, leachable solvents and bioactive drugs are proposed to be delivered adjacent to root surface in periodontal defects for stimulating periodontal regeneration and suppressing the inflammation associated with the healing response. However, the composition is semi-solid and hardens upon placement, while lacking any defined microarchitecture for cell guidance. As such, the invention is more related to drug delivery in periodontal defect sites.

U.S. Pat. No. 5,455,041; “Method for inducing periodontal tissue regeneration”, (Authors: Genco R J, Cho M I; Publication Date: Sep. 9, 1995). The application of growth factors following treatment of tooth root surface with demineralizing agents as part of guided tissue regeneration procedure is described in this invention. The applied growth factors may influence the resident cells in and around the periodontal defect site, but the lack of scaffold does not allow for controlled release and cell guidance by provisional extracellular matrix structure.

U.S. Pat. No. 4,961,707; “Guided periodontal tissue regeneration”, (Authors: Magnusson I, Batich C; Publication Date: Oct. 9, 1990). The invention is related to the bioabsorbable synthetic and natural materials with a porous structure and being able to carry biologically active factors for covering the exposed tooth root surface during periodontal surgical application. However, the structure is aimed to function as a barrier to prevent gingival epithelial and fibroblastic cells' migration into the periodontal defect site, thereby allowing the spontaneous regeneration by periodontal ligament-based regenerative cells. Thus, the patent is reflecting the “guided tissue regeneration” (GTR) approach known and applied in the field of periodontal surgery by those skilled in the art for more than two decades and does not provide scaffold for the regenerative cells of periodontium, as was evident also from the application methods of the subject of the invention.

U.S. Pat. No. 5,197,882; “Periodontal barrier and method for aiding periodontal tissue regeneration agents”, (Author: Jernberg G R; Publication Date: Mar. 30, 1993).

This patent is also related to the barrier membranes incorporating chemotherapeutic agents for controlled and/or cyclical release. The membrane and its application also conform to the principle of the GTR, but not the tissue engineering strategy based on the induction of multiple activities of regenerative cells in a multifaceted combinatorial approach.

U.S. Pat. No. 5,656,593; “Morphogen induced periodontal tissue regeneration”, (Authors: Kubersampath T, Rueger D C, Oppermann H, Cohen C M, Pang R H L; Publication Date: Aug. 12, 1997). This patent describes morphogens such as dimeric proteins and their application methods into periodontal tissues to repair and regenerate lost structures or inhibit such a loss. Although the morphogens are aimed at stimulating cementoblastic and fibroblastic cells, and can be applied with acellular matrix, the precisely defined microarchitectural features that will affect cellular behavior is not present and the effects are based on the actions of the applied morphogenic molecules.

U.S. Patent No. 2006/0188544; “Periodontal tissue regeneration using composite materials comprising phosphosphoryn”, (Author: Saito T; Publication Date: Aug. 24, 2006). Composite biomaterials comprised of phosphosphoryn and collagen and having porous structure for periodontal regeneration have been described. The structures may contain bone marrow-derived cells to aid the regeneration. Lacking the external stimuli of various growth factors, the approach seems to be more useful as osteoconductive vehicle in alveolar bone defects.

U.S. Pat. No. 5,885,829; “Engineering oral tissues”, (Authors: Mooney D J, Rutherford B R; Publication Date: Mar. 23, 1999). The invention provides methods for engineering various oral tissues from viable cells using ex vivo culture on a structural matrix. The requirement for cell-containing tissue harvesting and ex vivo culture is obvious, representing a different approach from in situ cell-induction with tissue engineering three-dimensional scaffolds possessing defined architecture and bioactive molecular composition.

E.U. Patent No. EP1272127; “Methods for production of ligament replacement constructs”, (Authors: Laurencin C T, Frank C O, Cooper J A, Helen L U H, Attawia M A; Publication Date: Dec. 26, 2007). This invention provides methods for fabrication of three dimensional scaffolds from biodegradable polymers with braiding techniques, to be seeded with anterior cruciate ligament fibroblasts and used to regenerate the damaged skeletal ligament structures. The mechanical properties of the scaffolds are emphasized with a focused regeneration on load bearing structures such as anterior cruciate ligament. The characteristics of the invention do not represent optimal system for the regeneration of periodontal structures.

U.S. Patent No. 2007/0259018 A1; “Implant depots to deliver growth factors to treat avascular necrosis”, (Author: McKay W F; Publication Date: Aug. 11, 2007). This invention is related to a design and composition of depot implant for the delivery of growth factors to induce angiogenesis in avascular bone tissue. The delivery vehicle is natural or synthetic polymer, but the porous architecture is designed with the aim of growth factor seeding and diffusion, thereby representing a drug delivery device for the treatment of avascular necrosis in mineralized tissues.

U.S. Patent No. 2008/0280360 A1; “Method for producing biomaterial scaffolds”, (Authors: Kaplan D L, Wong P Y; Publication Date: Nov. 13, 2008). The invention is related to multilayer scaffolds containing macro- and microchannels in different layers for tissue engineering. The structure is coated with bacterial cellulose, and may contain cells. However, defined bioactive agents for influencing resident cells in the vicinity of the scaffold are not provided. The invention is rather related to the multilayered constructs.

The state of the art in the field of periodontal regeneration entails the use of growth factors, barrier membranes and various bioactive agents and the vehicles for their delivery and release, however for the first time an integrated approach of multiple growth and differentiation factors combination at optimized concentrations, incorporated in tissue engineering scaffolds with nano-, micro-, and macroarchitectural properties that will serve as guiding clues for regenerative cells' migration, proliferation, differentiation and functional organization for the complete and consistent regeneration of diseased, defected and lost ligament structures, particularly that of periodontal ligament attachment apparatus, along with the methods for fabrication and application of such tissue engineering cell-guiding scaffolds is provided within the scope and utility of the present invention.

### DISCLOSURE OF INVENTION

### Summary

The subject of the present invention entails the use of the biomaterials, polypeptide growth and differentiation factors, chemoattractants and cell inducers to fabricate connective tissue engineering scaffolds for the regeneration of ligament and tendon tissues including but not limited to periodontal ligament, muscle-bone interface tendons, cranial bone interface sutures, skeletal joint ligaments, and intra- and periarticular tendons and ligaments and the tissue engineering-based surgical treatment of the defects of the tissues thereof. The biomaterials to be used are biocompatible and biodegradable polymers of natural and/or synthetic origin. The biomaterials can be selected from a group of synthetic polymers consisting of but not limited to poly(lactic acid), poly(glycolic acid), poly(lactic-co-glycolic acid), poly(ε-caprolacton), poly(vinyl alcohol) either as a single polymer type or as their combination; a group of natural biopolymers from the group consisting of but not limited to fibrinogen, fibrin, hyaluronic acid, collagen type I, collagen type III, fibronectin, laminin, vitronectin, gelatin, elastin, alginate and silk fibroin and the synthetic/natural biopolymers combination thereof. A particular embodiment in the present invention relates to the fabrication of the cell-guiding membrane scaffolds by methods of solvent-casting and porogen-leaching, phase separation and freeze-drying (lyophilization), rapid prototyping, and computer assisted solid free-form fabrication, such that renders the scaffolds with a microarchitecture of aligned and/or non-aligned intersecting and interconnecting channels and pores, rendering the said scaffolds with a high surface to volume ratio. In one of the embodiments, the said cell-guiding fibroinductive and angiogenic scaffolds' surfaces are precoated with extracellular matrix components such as laminin, fibronectin and collagen type I as cell attachment enhancing agents. The said extracellular matrix components are utilized to facilitate the initial attachment of the regenerative cells on the scaffold surfaces following in vitro seeding or more particularly in vivo implantation as part of a regenerative therapeutic procedure utilizing the said scaffolds. In a particular embodiment of the invention, the incorporation of polypeptide growth factors in an optimized concentration and combination into the cell-guiding scaffolds stimulating the chemotaxis, migration and proliferation of regenerative progenitor cells from the adjacent healthy tissues into the scaffolds, inducing the differentiation and supporting the maintenance of viability and functionality of the cells, thereby enhancing the formation of new regenerated tissues is disclosed. Particularly, the growth factors from the group consisting of basic fibroblast growth factor (bFGF), acidic fibroblast growth factor (aFGF), insulin-like growth factor-I (IGF-I), insulin-like growth factor-II (IGF-II)), platelet-derived growth factor αβ (PDGF αβ), platelet-derived growth factor ββ (PDGF ββ), brain-derived neurotrophic factor (BDNF), transforming growth factor-β (TGF-β), vascular endothelial growth factor (VEGF), bone morphogenetic protein-2 (BMP-2), bone morphogenetic protein-4 (BMP-4), and bone morphogenetic protein-7 (BMP-7) are used in combinations including some or all the factors in different and optimized dose and concentrations. The inductive effects of these growth factors on cellular proliferation, migration, viability, differentiation and functionality is widely demonstrated in scientific literature of the relevant fields and amplification of angiogenesis and osteogenesis through the endothelial and mesenchymal progenitor/stem cells' induction is achieved by their use. In another embodiment of the present invention the incorporation of somatotropin (growth hormone) to the cell-guiding scaffolds is described.

In another embodiment of the present invention the described aligned and/or non-aligned, interconnecting channels-containing and porous structure allows the cell migration inside the scaffolds and facilitates the development of fibrous connective tissue. Particularly, in periodontal ligament tissue regeneration the aligned channels are designed in specific regions of the scaffold to guide and support the cementoblastic and fibroblastic progenitors migration from alveolar bone perpendicularly towards tooth root surface, while in other regions to guide the similar cells from the healthy remaining periodontal ligament tissue from apical toward coronal direction parallel to the root surface. The interconnected porous structure is designed to support the osteoblastic progenitors and endothelial cell migration for bone formation and angiogenesis respectively.

The biomaterials of both synthetic and natural origin used in the fabrication of the scaffolds of the present invention that will serve as temporary constructs allowing regenerative cell attachment, spreading and organization during wound healing and tissue regeneration are also biodegradable, and following their guiding of the tissue pattern development are degrading both prior to and during the remodeling processes into their derivatives that are eliminated by the metabolic pathways of the organism. The effects of the cell-guiding fibroinductive and angiogenic scaffolds of the present invention on the human periodontal ligament cell attachment, migration, proliferation and extracellular matrix synthesis and deposition was demonstrated in in vitro experiments. The improved regeneration of experimental periodontal defects in animal models compared to the state-of-the-art regenerative techniques further proved the superiority of the cell-guiding fibroinductive and angiogenic scaffolds in periodontal tissue engineering-based regeneration. The cell-guiding scaffolds can be rendered with osteoinductive, fibroinductive, and/or cementoinductive properties according to the specific application requirements by manipulating the composition and concentration of the growth factors incorporated as well as nano-, micro and macroarchitectural properties of the said scaffolds.

Particularly, when the cell-guiding scaffolds are used in periodontal tissue engineering applications, the localization between tooth root surface and tissue-engineered or intact alveolar bone structure will allow the formation of functional ligament tissue between the mineralized tissues enabling maximum level of periodontal regeneration. The cell-guiding fibroinductive and angiogenic scaffolds of the present invention can be used to obtain superior periodontal regeneration results compared with the existing therapeutic modalities.

Unless otherwise defined, all technical and scientific terms used in the text have the same meaning as commonly understood by one of ordinary skill in the art to which this invention belongs.

The details of one or more embodiments of the invention are set forth in the following drawings and description below. Other features, objects, advantages and utilities of the invention will be apparent from the detailed description, examples, and from the claims.

### DETAILED DESCRIPTION OF THE INVENTION

The present invention discloses cell-guiding fibroinductive and angiogenic scaffolds for connective tissue engineering, the methods for their fabrication and the use thereof in guiding the regeneration of damaged tissues preferentially in ligament and membraneous structures such as but not limited to periodontal ligament, ligaments in joints such as the temporomandibular joint and joints of extremities, defects in periosteum of jaw bones, maxillofacial bones, cranial bones and skeletal bones, as well as cranial sutures in mammals and preferably humans. The underlying scientific rationale is based on the ability of these complex structures to stimulate the regenerative cells residing in the adjacent tissues to the defect site to migrate, proliferate, differentiate and function in a manner conductive for the regeneration of the absent structures, thereby restoring the morphology and function of the tissues aimed for the treatment. The amelioration of the intrinsic regenerative ability of the tissues is achieved by both eliminating the detrimental factors at the defect site prior to the application, and augmenting the regenerative cells' functions by the cell-guiding scaffolds in a manner conductive for superior regeneration that could not be attained by the spontaneous healing response of the organism.

Combining the tissue engineering approach of using biocompatible and biodegradable scaffolds as temporal templates for regenerating tissues and the activation of cell signaling mechanisms by extrinsic growth and differentiation factors and/or growth hormone, the cell-guiding fibroinductive and angiogenic scaffolds of the present invention affect the regenerative cells' activities at multiple levels such as adhesion, migration, proliferation, differentiation, and extracellular matrix components synthesis and secretion that play role in the complex and dynamic processes during wound healing, neotissue formation and tissue regeneration. The biodegradation kinetics of the materials (synthetic and natural polymers) that can be used in the fabrication of the scaffolds is widely demonstrated in the scientific literature and in the various clinical therapeutic applications. This degradation behavior renders the scaffolds the ability to serve as a temporal extracellular matrix for the cell attachment, migration and function at the initial phases of regenerative process, and gradually disappear as the new extracellular matrix is formed by the regenerative cells in the area.

The architectural features of the cell-guiding fibroinductive and angiogenic scaffolds contribute to the scaffolds' functions during tissue regeneration at several levels. The scaffolds mimic the natural extracellular matrix of the connective tissues with their nanotopological characteristics with nanofibrillar mesh structure and interconnected porous architecture rendering high surface-to-volume ratio. The phase-separation and freeze-drying steps during the synthetic polymer (PLLA, PLGA, PGA, etc.) scaffold fabrication are producing the nanofibrous structure with nanofibers ranging in diameters between 50-500 nm. The solvent-casting and porogen-leaching steps create interconnected macroporous network where pore size can be adjusted according to the chosen porogen diameters, which ranges between 50-500 μm, and more preferably between 100-300 μm in diameter. The variables in the fabrication parameters that affect mechanical and degradation properties, as well as porosity rate and polymer fiber diameter in the biodegradable synthetic polymer scaffolds are extensively described in the relevant fields of the scientific literature (Zhang R, M a XP. J Biomed Mater Res 2000; 52:430-8; Ma X P, Zhang R. J Biomed Mater Res 2001; 56:469-77). The resulting scaffold porosity rate of above 90% facilitates the cell migration and nutrient and metabolite mass transport, as well as accumulation of organized and functional cellular mass and extracellular matrix structures. The porous structure also enables the endothelial cell proliferation and sprouting angiogenesis being essential for viability of any tissue structure and the cells belonging to it (Kanczler J M, Oreffo R O. Eur Cell Mater. 2008; 15:100-14). Alternatively, the cell guiding scaffolds of the present invention can also be fabricated with techniques such as computer-assisted solid free-form fabrication and rapid prototyping. Such techniques for three-dimensional scaffold fabrication with predefined internal and external architectural features are well described in the art (Hutmacher et al. Trends Biotechnol. 2004; 22(7):354-62), and can be preferably used when the cell-guiding scaffolds are fabricated from synthetic polymers (for example PLLA, PLGA, PGA), rather than biopolymers of natural origin such as collagen type I and fibrin.

When biopolymers of natural origin (fibrinogen, collagen type I, etc.) are used in fabrication of the cell-guiding scaffolds of the present invention, the nanofibrous structure is also attained along with the superior cell attachment and proliferation-inductive abilities owing to the functional groups inherently present in the molecular structure of the natural biopolymers (Hubbell J A. Curr Opin Biotechnol. 2003; 14:551-8). Particularly, the polymerization of fibrinogen with thrombin leads to the formation of fibrin nanofibers and the mesh structure (blood clot) that is employed by the organism for the cessation of bleeding following injury and subsequently as wound healing vehicle that has multiple effects on cell adhesion, migration, proliferation and functional organization in neotissue formation (Laurens et al. J Thromb Hemost 2006; 4:932-9).

However, the nanofibrous structure with its interconnected porous network at nanometer level is not permissive for mammalian cell migration per se, owing to the difference of the cellular size and interfiber distances. While the nanofiber diameters can vary between 50-500 nm depending on the production parameters when synthetic polymers are used and in a similar range of natural collagen-based extracellular matrix and fibrin clot structures, the interfiber spaces are also in submicron range. Cellular dimensions however vary between 5-50 μm depending on the cell type, with fibroblastic, osteoblastic and cementoblastic progenitors and their progeny most commonly varying between 7-15 μm in diameter. Consequently the cellular size of the connective tissue cells is not conductive for unconstrained cell movement through the nanoporous spaces of nanofiber mesh scaffolds. In natural wound healing when fibrin clot is formed, the cells from the surrounding tissues invade the clot by degrading the matrix with the proteolytic enzymes such as matrix metalloproteinases, which cleave the fibrin molecules at specific sites inherently present for recognition of the given enzyme (Mosesson M W. J Thromb Hemost 2005; 3:1894-904). Thus, temporal extracellular matrix is degraded by cellular activity as immune system cells, fibroblasts and endothelial cells advance and gradually replace the provisional matrix with a neotissue formation. Meanwhile, the resulting fragments of the enzymatic cleavage of fibrin molecules act further to stimulate cell migration and proliferation, and bound growth factors and chemotactic molecules are freed gradually, thus eliciting their actions in a controlled way (Laurens et al. J Thromb Hemost 2006; 4:932-9). When biodegradable synthetic polymers such as but not limited to PLLA and PLGA are used for the fabrication of tissue engineering scaffolds with nanofiber architecture, they are not permissive for cell proliferation and migration through the nanoporous network of the randomly oriented fibrous non-woven mesh structure as was demonstrated experimentally (Telemeco et al. Acta Biomater. 2005; 1(4):377-85). The difference in comparison to the fibrin-based extracellular matrix is that although synthetic polymers are biocompatible and biodegradable, they lack the surface characteristics for cellular interaction and also different degradation kinetics and degradation products (lactic acid for example) are detrimental rather than stimulating to regenerative process directed by the progenitor cells in the defect site (Lee J W, Gardella Jr J A. Anal Bioanal Chem. 2002; 373:526-37).

The cell-guiding fibroinductive and angiogenic scaffolds of the present invention posses defined macroporous structure of interconnected channels with predesigned orientation, facilitating the cell migration in desired directions in natural as well as synthetic polymer-based structures. In a particular embodiment when the scaffolds are aimed for the use in periodontal regeneration, the scaffold surfaces that will face the tooth root and alveolar bone contain porous architecture permissive for the beginning of the transverse migration of regenerative cells through the scaffold thickness perpendicular to the root direction. The inner architecture of the cell-guiding scaffolds displays multiple interconnecting channels whose axial orientation and direction can be tailored according to the desired cellular movement directions. Particularly, the channels' axial direction can be vertical and parallel to the tooth root in the apical portion of the scaffolds, whilst gradually acquiring oblique to perpendicular orientation towards the coronal parts of the scaffolds. The apical portion of the scaffold in this instance refers to the part of the scaffold that will be close to the tooth root apex, and coronal portion refers to the part that will be close to tooth crown when the scaffold is placed in situ in periodontal defect for the regeneration of the lost periodontal structures. Hereby, the scaffolds' cell-guiding channel structure can be prepared with a variable axial orientation during the fabrication of the scaffold with the aim to ideally match the anatomical requirements of the teeth periodontia which are aimed for treatment. Thus, different cell-guiding scaffolds can be prepared depending on whether they will be used in incisors, canines, premolars and molar teeth of lower jaw or upper jaw, the number of roots and the anatomical organization of the healthy periodontal ligament fibers also being taken into consideration.

In one of the embodiments of the present invention the cell-guiding fibroinductive and angiogenic scaffolds' effect is the augmentation of the regeneration amount of the periodontal defects compared with the contemporary regenerative techniques available in the art. One of the means by which the cell-guiding scaffolds achieve this goal is the exertion of chemotaxis and stimulation of migration of the regenerative cells residing in the periodontal defects' neighboring healthy tissues by the bioactive substances incorporated into the scaffold structure, such as growth and chemotactic factors and/or growth hormone. The scaffold architecture of axially oriented channels system provides mechanical guide for the cellular movements as well as available spaces for the natural extracellular matrix that will be synthesized and deposited by these cells to replace the temporal tissue engineering matrix in the course of tissue regeneration process. Since the periodontal disease and destruction associated with it generally proceeds from coronal to apical direction along the tooth root, the periodontal defect is usually located on the coronal part of the periodontal structure and is bordered by a healthy remaining periodontal attachment apparatus on the apical part. The progenitor cells capable of regenerating the periodontal attachment structure by giving rise to cementoblasts, periodontal ligament fibroblasts and osteoblasts are thought to predominantly exist in this healthy remaining periodontal ligament and to a lesser degree in alveolar bone lacunae next to the defect site (Isidor et al. J Clin Periodontol. 1986; 13(2):145-50). The cell-guiding fibroinductive and angiogenic scaffolds exert their chemotactic and mitogenic effects on this remaining regenerative cell population, and the cell-guiding channels' direction in the apical parts of the scaffolds is preferably oriented oblique and parallel to the tooth root so as to allow the maximum regenerative cell migration in coronal direction to maximize the regeneration amount along the affected tooth root. Coronal portion of the cell-guiding scaffold on the other hand will be positioned next to cemento-enamel junction on the tooth root-crown boundary and this area is the final destination of the regenerative cells, where oblique and nearly perpendicular direction of the channels' axis to the root surface is aimed at facilitating the principal periodontal ligament fiber orientation in a similar way, recapitulating the original anatomical structure.

The interconnected nature of the channels' system present in the structure of the cell-guiding fibroinductive and angiogenic scaffolds of the present invention is developed also to allow the cellular interaction and neotissue continuity not only of the connective tissue-specific cells, but also the sprouting angiogenesis which is indispensible in supporting and maintaining the regenerative process and viability and functionality of all the cells in the area. In that regard, the present invention envisions the ample supply of blood vessel and capillary network in both the alveolar bone and healthy periodontal ligament tissue located adjacent to the defect site. For angiogenesis to occur, endothelial cell migration from the sprouting capillaries next to the scaffold is obligatory, and macroporous scaffold surface provides the necessary space for endothelial cell movement and advancing of the sprouting capillaries, whilst the interconnected nature of the channels system further support the vascularization throughout the scaffold. Nanoporous scaffold spaces on the other hand readily ensure the initial fluid movement throughout the structure, overcoming the mass transport and metabolite removal limitations that otherwise would be present parallel to the diffusion limitation in scaffolds with a thickness greater than a millimeter. Furthermore, as synthetic polymer scaffolds of the present invention will support the sprouting angiogenesis and mass transport by the macroporous and nanoporous architecture respectively, the natural biopolymer-based scaffolds such as but not limited to fibrin will additionally held the benefit of clot invasive properties of the connective tissue regenerative cells as well as endothelial cells.

In another embodiment of the present invention, the cell-guiding fibroinductive and angiogenic scaffolds exert their influences on regenerative cells' chemotaxis, migration, proliferation, differentiation and functional activities by the means of the different growth factors that are incorporated in the scaffold structure during the fabrication process. The growth factors are selected from the group consisting of basic fibroblast growth factor (bFGF), acidic fibroblast growth factor (aFGF), insulin-like growth factor-I (IGF-I), insulin-like growth factor-II (IGF-II), platelet-derived growth factor αβ (PDGF αβ), platelet-derived growth factor ββ (PDGF ββ), brain-derived neurotrophic factor (BDNF), transforming growth factor-β (TGF-β), bone morphogenetic protein-2 (BMP-2), bone morphogenetic protein-4 (BMP-4), bone morphogenetic protein-7 (BMP-7), and vascular endothelial growth factor (VEGF). The said growth factors can also be derived with a recombinant technology, thus being recombinant growth factors.

Having multiple effects on cellular functions widely demonstrated in the related scientific literature, the utilization of the aforementioned growth factors within the scope of the present invention is aimed at the exertion of chemotaxis to regenerative cells from the adjacent tissues of the defect site into and along the cell-guiding scaffolds, followed by the induction of cellular migration, proliferation, differentiation and synthesis and deposition of extracellular matrix components of the regenerating tissue of interest. The incorporation of the growth factors into the cell-guiding scaffolds can be performed with different methods known to those sufficiently skilled in the related art, and depends on the material properties chosen for the fabrication of the said scaffolds. Particularly, the growth factors can be loaded into synthetic as well as natural polymer scaffolds by impregnation, i.e. by incubation of the scaffolds in a solution containing one or more of the aforementioned growth factors in an appropriate impregnation buffer (e.g. phosphate buffered saline and other physiologic salt solutions). In another embodiment of the present invention, the chosen growth and differentiation factors can be incorporated into the scaffold by the means of intermediate protein, glycosaminoglycan and/or polysaccharide (for example: heparin) being irreversibly or reversibly connected to the scaffold matrix with a covalent or ionic bonding prior to the addition of the growth and differentiation factors. The covalent binding between the scaffold polymer and the intermediate protein (or glycosaminoglycan or polysaccharide) can be performed using for example amino-terminated PLGA in the fabrication of the scaffold and covalently binding the protein (or glycosaminoglycan or polysaccharide, for example heparin) to it through the reaction between the amino- and carboxylic acid groups utilizing standard carbodiimide chemistry (Jeon et al. Biomaterials 2007; 28:2763-71). Furthermore, the growth factors impregnation of the scaffolds of the present invention can be performed following the prior interaction between the linker agent (for example heparin) and the combination of the growth factors at predetermined concentrations.

The concentration of every growth and differentiation factor as well as somatotropin (growth hormone) can be adjusted according to the desired effects of the cell-guiding fibroinductive and angiogenic scaffolds. Particularly, the angiogenic effects are present in every type of the cell-guiding scaffolds of the present invention. This effect is ensured by the incorporation of the growth factors with widely demonstrated angiogenic activity, such as but not limited to vascular endothelial growth factor (VEGF), basic fibroblast growth factor (bFGF), and platelet-derived growth factor (PDGF) (PDGFαβ and PDGFββ isomers). In another aspect of the present invention, the concentration of the growth and differentiation factors incorporated in the cell-guiding scaffolds can be adjusted according to the tissue type that is intended to be induced, for example fibrous ligament connective tissue or cement or bone. Accordingly, the composition and concentration of bone-inductive or fibro-inductive growth and differentiation factors can be adjusted. Thus, when induction of mineralized tissue is aimed, all of the bone morphogenetic proteins (BMP-2, BMP-4 and BMP-7) will be incorporated at the optimal concentration for osteoinduction. The bFGF, PDGF (PDGFαβ and PDGFββ isomers) and IGF (IGF-I and IGF-II) will also be present as well as BDNF. Particularly, when the cell-guiding scaffolds of the present invention are fabricated with the intention of regeneration of periodontal defects, the differentiation factors with cementogenic properties can also be incorporated into the structures. These factors include but are not limited to the factors selected from the group of enamel matrix proteins consisting of amelogenin, ameloblastin, enamelin, amelotin, odontogenic ameloblast associated protein (ODAM), and also the dentin matrix proteins dentin-derived phosphosphorin (DPP) and dentin sialoprotein (DSP).

In a particular embodiment of the present invention, the concentration of the growth and differentiation factors incorporated in cell-guiding scaffolds can be adjusted according to the release kinetics influenced by the scaffold material, the presence of the intermediate binding agent (protein, glycoprotein, glycosaminoglycan, polysaccharide) and the type of the binding (covalent crosslinking or otherwise). According to a specific aspect of the embodiment, basic fibroblast growth factor (bFGF) concentration varies in the range of 1 μg/ml-1000 μg/ml, and more preferably between 5 μg/ml-500 μg/ml, and still more preferably between 10 μg/ml-300 μg/ml. In a multicenter randomized double-blinded clinical study, the bFGF application to periodontal defects at concentration ranging between 300 μg/ml-3000 μg/ml resulted with a significant attachment gain and 2-fold bone height gain after 36 months postoperatively (Kitamura et al. PLoS ONE. 2008; 3(7):e2611.) According to another aspect of the invention, the concentration of acidic fibroblast growth factor varies in the range between 5 μg/ml-500 μg/ml, and more preferably between 10 μg/ml-300 μg/ml. According to still another aspect of the invention, the concentration of platelet-derived growth factor (PDGF-all isoforms used) varies between 1 μg/ml-1000 μg/ml, and more preferably between 5 μg/ml-500 μg/ml, and still more preferably between 10 μg/ml-300 μg/ml. In a randomized, triple-blinded multicenter clinical trial, the PDGFββ application into periodontal defect sites at concentrations of either 300 μg/ml or 1000 μg/ml resulted in a significant attachment gain 3 months post-operatively, and 2.5-3 fold greater bone height and mass gain at both 3 and 6 months postoperatively (Nevins et al. J. Periodontol. 2005; 76(12):2205-15). Insulin-like growth factor (IGF-I and IGF-II isoforms) concentration range is preferably between 1 μg/ml-1000 μg/ml, and more preferably between 5 μg/ml-500 μg/ml, and still more preferably between 10 μg/ml-300 μg/ml. In a human clinical trial, the combined application of PDGFββ and IGF-I at concentrations either of 50 μg/ml or 150 μg/ml into intraosseous periodontal defects resulted with ˜2-fold greater bone filling at the defect sites 6 and 9 months postoperatively compared to controls, and without any side effects to the patients demonstrated with hematological and serologic tests (Howell et al. J. Periodontol. 1997; 68(12):1186-93). In a specific aspect of another embodiment of the invention, vascular endothelial growth factor (VEGF) can be incorporated into cell-guiding scaffolds at concentration ranging between 1 μg/ml-1000 μg/ml, and more preferably ranging between 5 μg/ml-500 μg/ml, and still more preferably ranging between 10 μg/ml-300 μg/ml. In another aspect of an embodiment of the invention, the brain-derived neurotrophic factor (BDNF) can be incorporated at concentration ranging between 1 μg/ml-1000 μg/ml, and more preferably ranging between 5 μg/ml-500 μg/ml, and still more preferably ranging between 10 μg/ml-200 μg/ml. According to another aspect of a particular embodiment of the present invention, the concentration of BMP-2 that can be incorporated into the cell-guiding scaffolds ranges between 1 μg/ml-1000 μg/ml, and more preferably between 5 μg/ml-500 μg/ml, and still more preferably between 10 μg/ml-200 μg/ml. Recombinant BMP-2 incorporated into poly(D,L-lactide-co-glycolide) scaffolds through P-dioxane or poly-ethylene glycol at concentration of 5-20 μg/ml resulted in ectopic bone-inducing activity greater than allogenic demineralized freeze-dried bone allografts (Sato et al. Nat. Biotechnol. 2001; 19(4):332-5). According to still another aspect of the invention the concentration of BMP-4 that can be incorporated into the cell-guiding scaffolds ranges between 5 μg/ml-500 μg/ml, and more preferably between 10 μg/ml-200 μg/ml. In a further aspect, the concentration of BMP-7 that can be incorporated ranges between 5 μg/ml-500 μg/ml, and more preferably between 10 μg/ml-200 μg/ml.

In a particular embodiment of the present invention, the growth and differentiation factors can be incorporated into the cell-guiding scaffolds by means of impregnation in a solution containing said growth factors in a desired concentration, or chemically bound to the scaffold material by means of cross linking. More preferably in the context of the present invention, the growth and differentiation factors can be incorporated to the cell-guiding scaffolds using an intermediate binding agent (protein, glycoprotein, glycosaminoglycan, or polysaccharide) that will bind to both the growth and differentiation factors and the scaffold biomaterial. In a specific aspect of the present invention, the intermediate binding agent is preferably heparin, wherein said growth factors are bound to heparin prior to the incorporation into the scaffold or are incorporated to the scaffold already containing the intermediate binding agent (i.e. heparin). In another aspect of the present invention, the heparin-bound growth and differentiation factors are incorporated into the said cell-guiding scaffolds that are preferably fabricated from the fibrin polymer. In a particular embodiment of the present invention, the growth and differentiation factors-incorporated cell-guiding scaffolds are preferably processed so as to ensure the stabilization of the said growth factors inside the said scaffolds. Preferably, said stabilization is achieved by the method of freeze-drying, resulting with the dry complex of scaffold containing growth and differentiation factors.

Another embodiment of the present invention envisages the incorporation of growth and differentiation factors into the cell-guiding scaffolds such as to achieve a concentration gradient inside the scaffold. The said concentration gradient can be present throughout the thickness, and more preferably throughout the length of the scaffold. Furthermore, the concentration gradient can be applied to one or more growth and differentiation factors chosen to be incorporated into the scaffold structure. Particularly, when periodontal regeneration is aimed, the concentration gradient along the length of the scaffold can be applied so as to increase or decrease in coronal direction. The said concentration gradient effects can be aimed at cell migration, cell proliferation, cell differentiation and cellular functions of extracellular matrix components synthesis and secretion, depending on the defect type and characteristics and the anatomical features of the specific localization in the body. In a particular embodiment, the concentration gradient can be achieved by growth and differentiation factors incorporation at different concentrations into the different regions of the cell-guiding scaffolds during the fabrication process. Alternatively, such a gradient can be achieved by fabricating separate scaffold segments with different concentrations of growth and differentiation factors, followed by the assembling of the segments with the same biomaterial (for example fibrin cell-guiding scaffold segments connection by additional fibrin polymerization between the separate parts). Such a gradient can vary between 0.1 to 100 times, and more preferably between 0.5 to 10 times of a given concentration along the entire length of the cell-guiding scaffolds.

Another embodiment of the present invention envisages the incorporation of somatotropin (growth hormone) into the cell-guiding scaffolds. The somatotropin can be utilized in instances including but not limited to the tissue defects requiring extensive cellular proliferation to achieve the satisfactory regeneration. Advanced periodontal defects where most of the periodontal ligament tissue is lost, articulate and skeletal joints' ligament injuries and bone defects accompanied by the extensive loss of the periosteum are some of the non-limiting examples representing such instances. In another embodiment of the present invention, somatotropin (growth hormone) can be incorporated into the cell-guiding scaffolds by means of impregnation (for example in a buffer solution such as phosphate buffered saline), or by binding through intermediate protein (or glycoprotein, glycosaminoglycan, polysaccharide) acting as a linker between the said hormone and the scaffold biomaterial. In another embodiment of the present invention, the somatotropin (growth hormone) can be incorporated into the cell-guiding scaffolds in a concentration gradient varying between 0.1-100 times, and more preferably between 0.5-10 times along the length of the said scaffolds. Such a concentration gradient can be achieved by somatotropin (growth hormone) incorporation at different concentrations into the different regions of the cell-guiding scaffolds during the fabrication process. Alternatively, such a gradient can be achieved by fabricating separate scaffold segments with different concentrations of growth hormone, followed by the assembling of the segments with the same biomaterial (for example fibrin cell-guiding scaffold segments connection by additional fibrin polymerization between the separate parts).

In a particular embodiment of the present invention, the cell-guiding fibroinductive and angiogenic scaffolds' surfaces were coated with proteins enhancing initial attachment and adhesion of the regenerative cells. Preferably the said proteins are natural extracellular matrix proteins such as but not limited to fibronectin, laminin and collagen type I. Within the scope of the present invention, the said cell-guiding scaffolds are covered with the said cell-adhesion facilitating proteins following scaffold preparation and before growth and differentiation factors incorporation into the scaffolds. Additionally or alternatively, the said cell-adhesion facilitating proteins can be incorporated into the cell-guiding scaffolds following the incorporation of the growth and differentiation factors. Within the scope of the present invention, when the cell-adhesion facilitating proteins are incorporated into the cell-guiding scaffolds, it is performed prior to the utilization of the scaffolds in animal and/or human subjects as part of the regenerative therapies.

In another embodiment of the present invention, when it is aimed to render osteoinductive properties to the cell-guiding scaffolds, the inorganic substances such as but not limited to calcium carbonate, calcium phosphate, hydroxyapatite and nanohydroxyapatite crystals can additionally be incorporated into the scaffold structure as osteoconductive agents. Specifically within the scope of the present invention, the incorporation of the osteoconductive inorganic substances is envisioned when the cell-guiding scaffolds are to be placed in contact with a bone surface during the regenerative treatment procedures utilizing the said scaffolds. Particularly, when the cell-guiding scaffolds are used in periodontal regeneration, the said bone surface is the surface of the intact alveolar bone in the periodontal defect or the surface of the bone tissue engineering scaffolds placed into the defect. The incorporation of the said inorganic substances to the said scaffold structure can be performed during the scaffold fabrication process. Alternatively, the incorporation of the said inorganic substances to the scaffold structure can be performed following the fabrication process and prior to the incorporation of the cell-adhesion facilitating proteins, growth and differentiation factors and/or growth hormone to the said cell-guiding scaffolds. Alternatively, the osteoconductive inorganic substances can be incorporated into the cell-guiding scaffold structure between the cell-adhesion protein coating and growth and differentiation factors incorporation steps. In a specific aspect of the embodiment, the osteoconductive inorganic substances can be incorporated into the cell-guiding scaffolds so as to be present only in a portion of the scaffold or preferably localized in one of the surfaces of the scaffold. The said portion or surface in this instance refers to the part of the cell-guiding scaffold that will localize next to the bone surface when scaffold is placed in a defect site intended for treatment. The osteoconductive properties of the said inorganic substances embodied herein are well known in the relevant art and are extensively used both in experimental and clinical therapeutic bone tissue regenerative and reparative procedures (LeGeros R Z. Clin Orthop Relat Res. 2002; 395:81-98). The calcium phosphate in the form of tricalcium phosphate is widely used, and hydroxyapatite is the natural crystal structure of the inorganic component of the human bone tissue.

In another embodiment of the present invention, the cell-guiding fibroinductive and angiogenic scaffolds can be sterilized following the fabrication process with techniques such as but not limited to gamma-irradiation, ethylene oxide sterilization and ethyl alcohol sterilization. Another aspect of the invention envisages the incorporation of the growth and differentiation factors and cell-attachment facilitating proteins into the cell-guiding scaffolds as sterile solutions by means of the dissolving the said growth and differentiation factors and cell-attachment facilitating proteins in pre-sterilized buffer solutions (for example phosphate buffered saline), or the sterilization of the said solutions by means such as filtration following the dissolution of the said factors and proteins.

### BRIEF DESCRIPTION OF DRAWINGS

FIG. 1. Schematic description of the preparation of fibroinductive and angiogenic cell-guiding scaffolds with synthetic polymers (PLLA and/or PLGA) by the solvent-casting and porogen-leaching, and freeze-drying methods. (A) Dispersing of (1) salt (NaCl) crystals and (2) assembled sugar-based fiber meshes in a (3) casting mold; (B) Casting the dissolved (4) synthetic polymer solution into the mold. (C) Dissolving of the (5) porogen components in (6) distilled water after polymerization. (D) Fabrication of the cell-guiding scaffolds with phase-separation and freeze-drying methods. Phase-separated and porogen-leached (7) polymer gel scaffold in the (8) freeze-dryer.

FIG. 2. Schematic description of the preparation of fibroinductive and angiogenic cell-guiding scaffolds with fibrinogen/fibrin by solution-casting, polymerization, and porogen-leaching methods. (A) Dispersing of poly(methyl methacrylate) (PMMA) (8) particles and (9) fiber meshes in a (10) casting mold; (B) Casting the (11) fibrinogen solution into the mold. (C) Polymerization with (12) thrombin and CaCl₂; (D) Dissolving and elimination of the porogen components from the scaffold structure in (13) organic solvent (acetone).

FIG. 3. Fibronectin and laminin incorporation into the cell-guiding scaffolds fabricated from both natural and synthetic polymers for enhancing cellular adhesion (A). (14) Cell-guiding scaffold; (15) fibronectin-laminin mixture. Growth factors adsorption into the fibrin based cell-guiding scaffolds by using heparin (B). (16) Mixture of growth factors combination at optimized concentration; (17) heparin-bound growth factors mixture; (C) incorporation of heparin-bound growth factors solution into cell-guiding fibrin scaffolds. (D) freeze-drying of (18) heparin-bound growth factors-containing cell-guiding fibrin scaffolds.

FIG. 4. Schematic representation of the cell proliferation and/or migration experiments with human periodontal ligament fibroblastic cells (hPDLF) inside the cell-guiding fibroinductive and angiogenic fibrin scaffolds in vitro. (A) Transverse migration experiments following hPDLF cell seeding on the cell-guiding scaffold surface. (18) cell-guiding scaffold; (19) micropipette tip; (20) hPDLF cells; (21) cell culture coverslip. (B) Longitudinal migration experiments in the cell-guiding scaffolds. (18) cell-guiding scaffold; (19) micropipette tip; (20) hPDLF cells; (22) line of semi-separation.

FIG. 5. Graphical presentation of the quantitative results following cell proliferation experiments with human periodontal ligament fibroblastic cells (hPDLF) inside cell-guiding scaffolds in vitro. hPDLF cell proliferation inside the unmodified cell-guiding PLGA (CGS(PLGA)), unmodified cell-guiding fibrin (CGS(Fibrin)), cell-guiding fibroinductive and angiogenic PLGA (FIA-CGS(PLGA)), cell-guiding fibroinductive and angiogenic fibrin (FIA-CGS(Fibrin)), cell-guiding osteoinductive and angiogenic fibrin (OIA-CGS(Fibrin)), and cell-guiding cementoinductive fibrin (CI-CGS(Fibrin)) scaffolds. Data represent mean values±SD of 5 independent experiments as cell proliferation %

$$( \frac{proliferating cells}{non \text{-} proliferating cells} \times 100 ) ;$$

statistical significance is set at p<0.05.

FIG. 6. Graphical presentation of the quantitative results following cell migration experiments with human periodontal ligament fibroblastic cells (hPDLF) inside cell-guiding scaffolds in vitro. Cellular migration is measured in longitudinal direction inside unmodified cell-guiding PLGA (CGS(PLGA)), unmodified cell-guiding fibrin (CGS(Fibrin)), cell-guiding fibroinductive and angiogenic PLGA (FIA-CGS(PLGA)), and cell-guiding fibroinductive and angiogenic fibrin (FIA-CGS(Fibrin)) scaffolds. Data is presented from three independent experiments as millimeter cell migration distance along the longitudinal direction of the scaffolds, and is expressed as mean±SD; statistical significance is set at p<0.05.

FIG. 7. Schematic representation of the experimental periodontal defects in dog's mandibular 3^{rd} and 4^{th} premolar teeth and their treatment with cell-guiding scaffolds. (A) Dog's mandibular 3^{rd} and 4^{th} premolar teeth root structure. (23) 3^{rd} premolar tooth; (24) 4^{th} premolar tooth. (B) Exposed root surfaces after flap removal. (25) mucoperiosteal flap; (26) line of vertical incision. (C) Application of cell-guiding fibroinductive and angiogenic, osteoinductive and angiogenic or cementoinductive scaffolds on the 3^{rd} and 4^{th} mandibular premolars following surface debridement. (27) cell-guiding fibroinductive and angiogenic, osteoinductive and angiogenic, or cementoinductive scaffolds. (D) Covering of defects treated with cell-guiding scaffolds with barrier membranes (ePTFE) to prevent epithelial and gingival fibroblastic cell migration into the defect area. (28) barrier membrane (ePTFE). (E) Closing and suturing of soft tissues over the treated area. (29) interrupted sutures.

FIG. 8. Graphical presentation of new cementum, new alveolar bone, new periodontal ligament attachment gain in experimental periodontitis defect models in dog third and fourth premolar teeth as a result of implantation of cell-guiding fibroinductive and angiogenic, osteoinductive and angiogenic, and cementoinductive fibrin scaffolds. (A) postoperative 4^{th} week measurements in % defect length, and (B) postoperative 12^{th} week measurements in % defect length. Data represent mean values±SD from 20 specimens from every condition and time point. Statistical significance set at p<0.05.

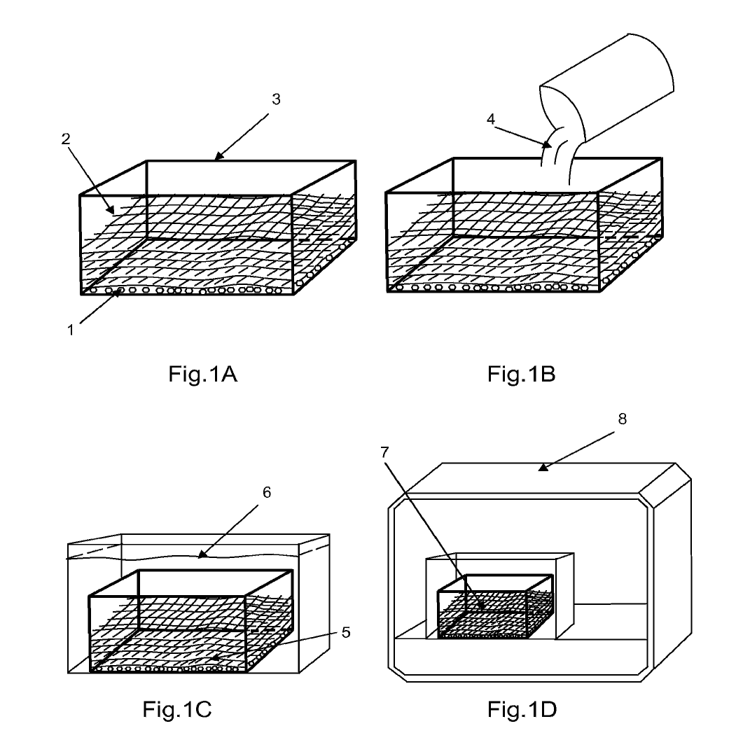

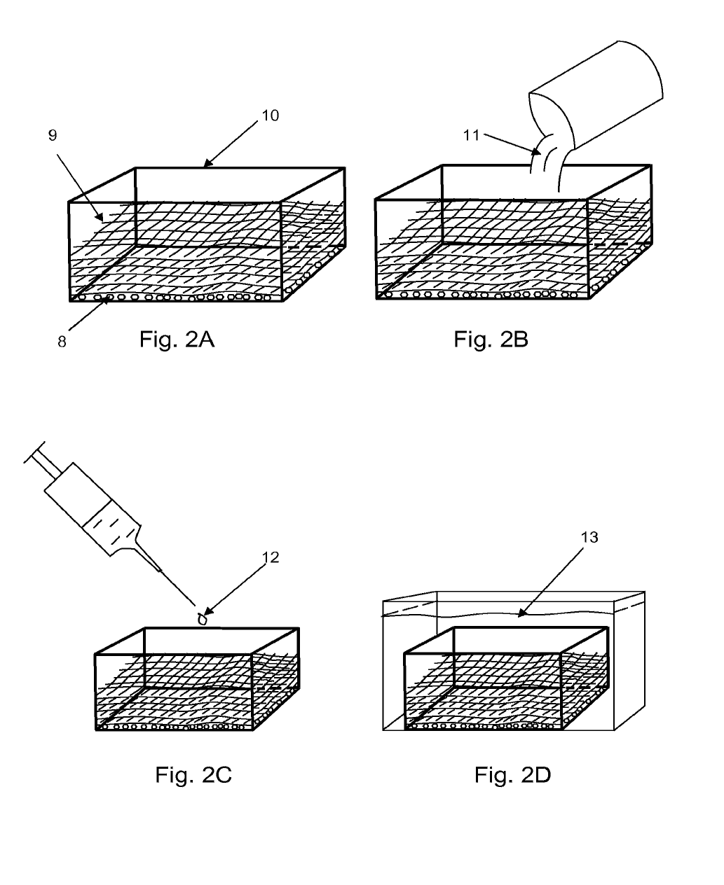

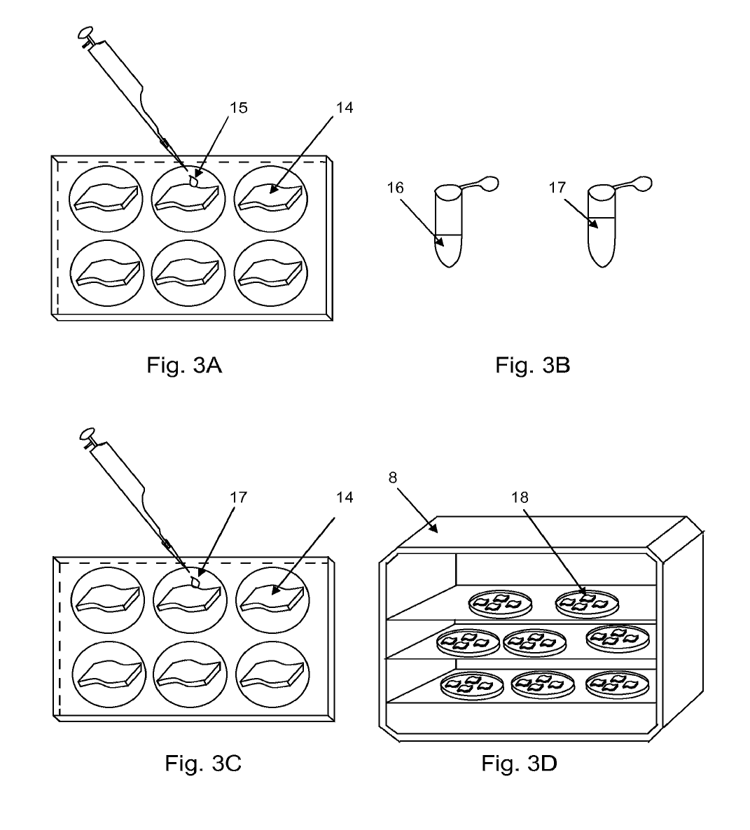

### BEST MODES FOR CARRYING OUT THE INVENTION

In a particular embodiment, the present invention envisages the utilization of the cell-guiding fibroinductive and angiogenic scaffolds in in vitro cell-adhesion experiments. Such experiments are envisioned to provide scientific data on the effects of the said scaffolds on cell adhesion of various tissue cells possessing the potential to play a role in connective tissue regeneration such as but not limited to fibroblasts, osteoblasts, cementoblasts, osteoprogenitor cells, adult tissue-specific stem cells, bone marrow-derived mesenchymal stem cells, pluripotent embryonic stem cells, as well as angiogenic cells such as endothelial cells and smooth muscle cells.

In another embodiment, the present invention envisages the utilization of the cell-guiding scaffolds in in vitro cell-migration experiments. Within the scope of this invention, such experiments are envisioned to provide scientific data on the effects of the said scaffolds on cell migration of various tissue cells possessing the potential to play a role in connective tissue regeneration such as but not limited to fibroblasts, osteoblasts, cementoblasts, osteoprogenitor cells, adult tissue-specific stem cells, bone marrow-derived mesenchymal stem cells, pluripotent embryonic stem cells, as well as angiogenic cells such as endothelial cells and smooth muscle cells. Particularly, such experiments can be performed to elucidate the effects of the nanotopological, microtopological and macrotopological characteristics of the cell-guiding scaffolds, their macroporous and interconnected channels-containing inner architecture and the incorporated growth and differentiation factors and growth hormone combination, concentration and concentration gradients on the chemotactic and cell migration enhancing effects of the said scaffolds at the cellular and molecular biologic levels. In another embodiment, the cell-guiding scaffolds of the present invention can be utilized in cell differentiation experiments in vitro with the aim of studying the effects of the said cell-guiding scaffold composition (growth and differentiation factors content, concentration, and combination) on the differentiation induction of progenitor and stem cells such as but not limited to periodontal ligament progenitor cells, osteoprogenitor cells, adult tissue-specific stem cells, neural crest cells, bone marrow-derived mesenchymal stem cells, and undifferentiated pluripotent embryonic stem cells. More particularly, the said experiments can be performed with the aim to study the effects of various growth and differentiation factors concentration and combination on the differentiation induction and lineage selection of undifferentiated and partially differentiated cells at the cellular, genetic and molecular biologic levels.

In another embodiment of the present invention, the utilization of the cell-guiding fibroinductive and angiogenic scaffolds to determine the effects on extracellular matrix synthesis and deposition by the connective tissue cells and angiogenic cells in vitro is envisioned. Particularly, the obtaining of experimental data on regenerative processes at cellular, genetic and molecular biologic levels in periodontal ligament fiber formation, Sharpey's fiber formation, cementogenesis, osteogenesis, angiogenesis in ligaments and periosteum is envisioned within the scope of the present invention.

In a particular embodiment of the present invention, the cell-guiding fibroinductive and angiogenic scaffolds can be seeded with one or more of the cells from the group including but not limited to autologous periodontal ligament stem cells, cementoblastic cells, osteoblastic cells, osteoprogenitor cells, bone marrow-derived mesenchymal stem cells, adipose tissue-derived stem cells, dental follicle stem cells, human embryonic stem cells, and genetic engineered cells in vitro prior to the application as a therapeutic vehicle in tissue engineering of ligaments, tendons, and periosteum. Particularly, such in vitro cell-seeding can be followed by a culture period ranging from 1 to 21 days before the in situ implantation of the cell-guiding scaffolds as therapeutic vehicle, or alternatively the cell-seeded scaffolds can immediately be implanted to animal subject or human patient with a surgical procedure appropriately selected for a desired treatment.

In a particular embodiment of the present invention, the cell-guiding fibroinductive and angiogenic scaffolds' utilization for the regeneration of injured, diseased and destructed ligament tissues such as but not limited to periodontal ligament, temporomandibular joint ligaments, anterior cruciate ligament and joint ligaments throughout the skeletal system, as well as defects associated with the periosteum covering the cranial and skeletal bones is envisioned. Particularly within the scope of the present invention, the fibroinductive cell-guiding scaffolds can be rendered with osteoinductive and/or cementoinductive properties depending on the utility of the particular application. The said scaffolds' tissue-inductive properties can be adjusted with the utilization of the various biomaterials for scaffold fabrication (synthetic or natural polymers and their combination), various concentration, concentration gradient and combination of the growth and differentiation factors incorporated into the said scaffolds and various architectural properties at the nano-, micro- and macroscopic levels during the fabrication process.

In a particular embodiment, the cell-guiding fibroinductive and angiogenic scaffolds can be used in the regeneration of the periodontal defects in mammals, and particularly humans. The periodontal destruction can be the result of inflammatory periodontal diseases, tissue atrophy due to age and/or excessive functional loading, mechanical injury and iatrogenic factors. It is particularly envisioned within the scope of the present invention to apply the cell-guiding fibroinductive and angiogenic scaffolds during a periodontal surgical operation for the regeneration of the periodontal structures. The said scaffolds preferably can be placed on the tooth root surface exposed by the surgical procedure, after removal of the pathological debris (for example subgingival calculus, necrotic cementum, bacterial deposits) with the debridement of the root surface. The application of contemporary regenerative vehicles such as but not limited to barrier membranes, platelet-rich plasma and enamel matrix-derived proteins may or may not accompany cell-guiding fibroinductive and angiogenic scaffolds' utilization. In another embodiment, the using of a biological fixation agent such as fibrin glue for the stabilization of the cell-guiding scaffolds of the present invention is disclosed. The fibrin glue can be applied on the tooth root surface or alveolar bone surface corresponding to the external boundaries of the cell-guiding scaffold margins or the entire contacting surface of the root. Additionally or alternatively, the fibrin glue can also be used to connect the cell-guiding scaffold with a bone graft material or an osteoinductive scaffold positioned next to the scaffold of the present invention with the aim of regenerating the bone defect component of the affected periodontium.

In another embodiment, the cell-guiding scaffolds of the present invention can also be used in the regeneration of the ligament tissues such as but not limited to joint ligaments of knee, ankle, elbow, wrist, intervertebral ligaments, temporomandibular joint ligaments, and also periosteum of the cranial and skeletal bones being lost or injured by a disease processes, trauma, atrophy, iatrogenic factors or absent due to the congenital anomalies.

To further illustrate the features, objects and advantages of the present invention, the following non-limiting examples are presented. It will be apparent to those skilled in the art to which this invention belongs, that the similar or equivalent methods, materials and techniques can be used to fabricate, apply and modify the cell-guiding scaffolds of the present invention without departing from the scope and the spirit of the present invention. The contents of all references and published patents and patent applications, cited throughout this application are hereby expressly incorporated by reference.

### EXAMPLES

### Example 1

A. Fabrication of Cell-Guiding Fibroinductive and Angiogenic Scaffolds from Synthetic Polymers.

### 1) Preparation of Polymer Solutions

Two types of synthetic polymers from alpha-hydroxy ester group, poly(L-lactic acid) (PLLA) and poly(D,L-lactide-co-glycolide) (85/15)(PLGA85/15) (Sigma-Aldrich, St Louis, Mo.) were used to fabricate cell-guiding scaffolds. Both the PLLA and PLGA were dissolved in benzene or acetone (Sigma) at 60° C. in a magnetic stirrer for approximately 2 hours to obtain homogenous solutions of 5% (w/v).

### 2) Preparation of Porogen Replicas

Sodium chloride (NaCl) (Sigma) particles were sifted with sieves to obtain particles of two groups with sizes ranging between 100-250 μm and 250-500 μm. The sugar (sucrose, Sigma) particles were melted in a glass beaker at 120° C. A metal spatula was used to obtain fibers from the melted material as described (Zhang R, Ma X P. J Biomed Mater Res. 2000; 52:430-8). The tip of the spatula has been touched to the sugar melt and adhered to it. The tip has been moved away slowly to obtain a fiber that solidified at room temperature. The fiber diameter was determined by the drawing rate and speed. Fibers with diameters ranging between 100-300 μm were collected.

Synthetic polymer scaffolds fabricated by solvent-casting and porogen-leaching methods generally have nonporous layers on their lower surfaces which may hinder the cell migration and nutrient and metabolite transport. To prevent the formation of such a layer, the glass Petri dishes' (35×10 mm, Falcon) surface was covered with a thin layer of hydrophilic polymer (polypyrrolidone) (Sigma-Aldrich), which could be removed later by salt leaching. The polymer covered glass surface was covered with a single layer of NaCl particles of 100-250 μm size range with a sieve to obtain even distribution on the surface. Then, sugar fibers of approximately identical diameters (˜200 μm) were manually placed in parallel to each other forming the first layer. The second layer of fibers was laid on the top of the first layer with ˜30-35° angle of longitudinal axis orientation relative to the first layers' axis direction. The third layer was assembled in parallel to the first layer and with a 30-35° angle to the previous layers' orientation. Total of 5 to 8 of sugar fiber layers were assembled in a similar manner. The fiber assembly of each layer was stabilized by the water vapor treatment for 5-10 minutes, which resulted to adherence of sugar fibers in contacting points. The whole construct was kept in a moisturized atmosphere at room temperature for 30 minutes, followed by vacuum drying for 12 hours.

3) Preparation of Cell-Guiding Scaffolds with Nanofibrous, Macroporous and Channels-Containing Structure from Synthetic Polymers Using Solvent Casting and Porogen-Leaching, and Freeze-Drying Methods

Freshly prepared polymer solutions of 5% (w/v) PLLA or 5% (w/v) PLGA dissolved in benzene were slowly cast over the scaffold replica containing NaCl particles on the bottom and multiple layers of differentially oriented sugar fibers construct. The solution covered the entire construct, resulting in a thickness varying between 1000 to 1500 μm depending on the number of assembled sugar fiber layers. On top of the structure, NaCl particles of the same size as the bottom layer (100-250 μm) were evenly added as single layer cover. Both PLLA and PLGA constructs were kept in a freezer at −20° C. to allow the formation of gel and phase separation. Following the gelation and phase-separation procedure, the porogen-containing polymer gel structures were removed from the freezer and placed in a distilled water to simultaneously extract the remaining solvent and leach the salt particles and sugar fibers. The constructs were kept in distilled water for 3 days wherein water was changed every 6 hours. Following the leaching process, the gels were removed from the water and frozen for 3 hours in a freezer at −20° C. The structures were immediately placed into a freeze-dryer (Alpha 2-4 LSC, Martin Christ, Germany) and were freeze-dried at −20° C. under vacuum (pressure≦0.5 mmHg) for 10 days.

B. Fabrication of Cell-Guiding Fibroinductive and Angiogenic Scaffolds from Natural Biopolymers.

### 1) Preparation of Fibrinogen Solution

Human plasma fibrinogen (Sigma) was dissolved in water containing 0.9% NaCl at 37° C. for 2 hours at concentrations of either 150 mg/ml or 200 mg/ml. The completely dissolved fibrinogen solution was cast over the previously prepared replicas in Teflon molds.

### 2) Preparation of Porogen Replicas

Due to the dissolving properties of both NaCl and sugar-based porogen materials in aqueous solution used for the dissolution of fibrinogen, different materials dissolving in organic solvents were chosen as porogens for the formation of replicas. The poly(methyl methacrylate) (PMMA, Fluke) particles and fibers were used as described with some modifications (Linnes et al. Biomaterials 2007; 28:5298-306). The particles were sieved to obtain the size ranging between 100-250 μm. For fiber preparation, the PMMA was heated to 160° C. in a glass dishes until viscous melt was formed and the fibers were drawn by contacting a spatula to the surface of the melt and withdrawing it at varying speeds. The drawing rate and speed determines the fiber size, and the rapidly cooling and solidifying fibers were collected on a polytetrafluoroethylene (PTFE) plate surfaces. The fibers with diameters ranging between 200-300 μm were collected for replica formation. The bottom of the casting molds were covered with PMMA particles ranging between 100-250 μm such that a single layer of interconnecting particles was formed. Then, PMMA fibers of approximately identical diameters (˜200 μm) were manually placed in parallel to each other forming the first layer. The second layer of fibers was laid on the top of the first layer with ˜30-35° angle of longitudinal axis orientation relative to the first layer axis direction. The third layer was assembled in parallel to the first layer and with a 30-35° angle to the previous layers' orientation. Total of 5 to 8 of PMMA fiber layers were assembled in a similar manner. Then, the whole construct inside the mold was placed in an oven and heated to 145° C. for 22 hours as described (Linnes et al., Biomaterials 2007; 28:5298-306) to sinter the fibers and particles together, forming interconnected meshwork with three-dimensional configuration. The dimensions of the replicas were adjusted as ˜10×5×1 mm, ˜10×5×1.5 mm, or ˜20×5×1.5 mm.

3) Preparation of Cell-Guiding Scaffolds with Nanofibrous, Macroporous and Channels-Containing Structure from Fibrinogen-Fibrin System Using Solution-Casting, Protein Polymerization, and Porogen-Leaching Methods

The fibrinogen solution of either 150 μg/ml or 200 μg/ml was cast over the sintered and stabilized interconnected PMMA particles and three-dimensional oriented fiber mesh replica inside the PTFE molds. The solution covered the entire construct, resulting in a thickness varying between 1000 to 1500 μm depending on the number of assembled PMMA fiber layers. The mold was placed in a vacuum chamber for two hours (pressure of 50.5 mmHg) to ensure the complete infiltration of the fibrinogen solution into the voids of the PMMA replica. The infiltrated replicas were then removed from the vacuum chamber and the excess fibrinogen from the upper layer was gently scraped with sterile blades. The fibrinogen solution-containing replicas were transferred in 35-mm polystyrene cell culture dish in Dulbecco's Modified Eagle Medium (DMEM, Invitrogen, Carlsbad, Calif., USA) containing 14 U/ml Thrombin (Sigma) and 8.5 mM CaCl₂ (Sigma) and kept in room temperature for 16 hours for polymerization. The PMMA granular and interconnected fiber content was solubilized and removed by rinsing with series of acetone in an orbital shaker for 48 hours. To remove the acetone, the formed scaffolds were rinsed in orbital shaker in 100% ethanol, followed by rehydration by graded ethanol series with PBS (90%, 80%, 70%, and 60%). The scaffolds were finally rinsed with phosphate buffered saline (PBS) and kept in PBS at 4° C. until the modifications for cell attachment enhancement and growth and differentiation factors incorporation is performed. Some of the scaffolds were crosslinked with genipin (Sigma) to increase the stiffness of the scaffold structure. Following rehydration step with graded ethanol series, the scaffolds were kept in a 0.625% genipin solution in PBS for 12 hours. They were stored in PBS at 4° C. until further modifications.

### C. Enhancing Cell Adhesive Properties of Cell-Guiding Fibroinductive and Angiogenic Scaffolds.

In order to facilitate the initial cell attachment on the scaffold surfaces, cell-guiding scaffolds fabricated from both synthetic polymers (PLLA and PLGA) and natural biopolymers (fibrin) were precoated with natural extracellular matrix proteins known to enhance cell adhesion and attachment through interaction with integrin receptors on the cell membrane surfaces (Heino J, Kapyla J. Curr Pharm Des. 2009; 15(12):1309-17). For that purpose, fibronectin, laminin and collagen type I were selected for the present experiments. All of the used extracellular matrix (ECM) proteins were dissolved in PBS in following concentrations: human plasma fibronectin (20 μg/ml, Invitrogen, Paisley, UK); human placental laminin (10 μg/ml, Sigma); bovine collagen type I (30 μg/ml, Invitrogen). The synthetic polymer (PLLA and PLGA) cell-guiding scaffolds of predetermined dimensions (˜10×5×1 mm, ˜10×5×1.5 mm, or ˜20×5×1.5 mm) were placed in 50 ml centrifuge tubes (Corning, N.Y., USA) and 10 ml of fibronectin, laminin or collagen type I containing PBS solution was added. The scaffolds were kept in orbital shaker for 2 h in room temperature in fibronectin-containing solution for adsorption to take place. Laminin and collagen type I adsorption was performed at 37° C. for 24 hours in PBS containing 10 μg/ml and 30 μg/ml of laminin and collagen type I respectively. Some of the cell-guiding synthetic polymer scaffolds were incubated in PBS containing all tree extracellular matrix components at the aforementioned concentration, i.e. 30 μg/ml collagen type I, 20 μg/ml fibronectin, and 10 μg/ml laminin. The adsorption of combined ECM proteins was performed in 50 ml centrifuge tube on orbital shaker at room temperature for 24 hours. Following the adsorption procedures, the cell-guiding scaffolds were removed from the centrifuge tubes and washed with sterile PBS at room temperature in triplicate, then dried for 12 hours in a vacuum chamber.

The cell-guiding scaffolds fabricated from fibrin were also precoated with two ECM proteins, fibronectin and laminin. The adsorption of fibronectin was performed in a 10 ml PBS solution containing 20 μg/ml of fibronectin in 50 ml centrifuge tube on orbital shaker for 2 hours in room temperature. Laminin was adsorbed in a 10 ml PBS solution containing 10 μg/ml laminin in 50 ml centrifuge tube on orbital shaker at 37° C. for 24 hours. Some of the cell-guiding fibrin scaffolds were adsorbed with a combination of the laminin and fibronectin dissolved in PBS at 10 μg/ml and 20 μg/ml respectively at room temperature for 24 hours. Following adsorption, the cell-guiding scaffolds were rinsed with sterile PBS in triplicate and kept in PBS at 4° C. until growth and differentiation factors incorporation step.

D. Growth Factors Incorporation into the Cell-Guiding Fibroinductive and Angiogenic Scaffolds.
1) Growth Factors Incorporation into Synthetic Polymer Cell-Guiding Scaffolds

The incorporation of growth and differentiation factors in cell-guiding PLGA scaffolds was performed either following the extracellular matrix protein (fibronectin, laminin and collagen type I) adsorption or into the scaffolds without cell attachment facilitating modifications. Following the drying step, the ECM proteins-containing PLGA cell-guiding scaffolds were placed in 35 mm cell culture dishes. To render the cell-guiding scaffolds preferentially with fibroinductive and angiogenic properties, basic fibroblast growth factor (bFGF, human recombinant, Invitrogen), acidic fibroblast growth factor (aFGF, human recombinant, Invitrogen), insulin-like growth factor-I (IGF-I, human recombinant, Invitrogen), platelet-derived growth factor-ββ (PDGFββ, human recombinant, Invitrogen), and vascular endothelial growth factor (VEGF, human recombinant, Invitrogen) were reconstituted in sterile PBS at the following concentrations: bFGF (100 μg/ml), aFGF (20 μg/ml), IGF-I (50 μg/ml), PDGFββ (50 μg/ml), and VEGF (100 μg/ml) 100 μl from every reconstituted growth factor solution were withdrawn with sterile micropipette tips (DNase, RNase-free, Corning, N.Y., USA) and pooled in sterile 2 ml vial. The mixture was homogenized with vortex and then 500 μl of 8 IU/ml of heparin solution was added to the growth factor-containing mix. The final solution was homogenized at room temperature with vortex. The resultant 1 ml growth factor mix-containing heparin solution was immediately used for scaffold impregnation.

The incorporation of heparin-bound growth factors mix into the cell attachment facilitating ECM protein-coated PLGA cell-guiding scaffolds and non-treated PLGA scaffolds was performed in a similar way. The scaffolds were placed in wells of 12-well polystyrene cell culture plate (Nunc, N.Y., USA), one scaffold for each well. 50 μl of growth factor mix-containing heparin solution was transferred with a micropipette on the scaffold surface in a dropwise manner ensuring the even distribution of the solution on the entire scaffold surface. The ECM protein-adsorbed scaffolds were kept with the growth factors mix for 15 minutes at room temperature, while for the unmodified scaffolds the impregnation time was 45 minutes at the same conditions. Following the growth factor impregnation step, the cell-guiding scaffolds were placed into a freeze-dryer (Christ, Germany) and were freeze-dried at −20° C. under vacuum (pressure≦0.5 mmHg) for 10 days.

To render cell-guiding scaffolds preferentially with osteoinductive and angiogenic properties, basic fibroblast growth factor (bFGF, human recombinant, Invitrogen), insulin-like growth factor-I (IGF-I, human recombinant, Invitrogen), platelet-derived growth factor-ββ (PDGFββ, human recombinant, Invitrogen), bone morphogenetic protein-2 (BMP-2, human recombinant, Invitrogen), bone morphogenetic protein-4 (BMP-4, human recombinant, Invitrogen), bone morphogenetic protein-7 (BMP-7, human recombinant, Invitrogen), brain-derived neurotrophic factor (BDNF, human recombinant, Invitrogen) and vascular endothelial growth factor (VEGF, human recombinant, Invitrogen) were reconstituted in sterile PBS at the following concentrations: bFGF (100 μg/ml), IGF-I (50 μg/ml), PDGFββ (150 μg/ml), BMP-2 (50 μg/ml), BMP-4 (20 μg/ml), BMP-7 (50 μg/ml), BDNF (20 μg/ml) and VEGF (100 μg/ml). The incorporation of growth factors combination into the cell-guiding osteoinductive and angiogenic PLGA scaffolds was performed in the same way as described above.

To render the cell-guiding scaffolds preferentially with cementoinductive properties, basic fibroblast growth factor (bFGF, human recombinant, Invitrogen), insulin-like growth factor-I (IGF-I, human recombinant, Invitrogen), insulin-like growth factor-II (IGF-II, human recombinant, Invitrogen), platelet-derived growth factor-ββ (PDGFββ, human recombinant, Invitrogen), bone morphogenetic protein-2 (BMP-2, human recombinant, Invitrogen), bone morphogenetic protein-7 (BMP-7, human recombinant, Invitrogen), and brain-derived neurotrophic factor (BDNP, human recombinant, Invitrogen), were reconstituted in sterile PBS at the following concentrations: bFGF (100 μg/ml), IGF-I (50 μg/ml), IGF-II (50 μg/ml), PDGFββ (150 μg/ml), BMP-2 (50 μg/ml), BMP-7 (50 μg/ml), and BDNF (20 μg/ml). The incorporation of growth factors combination into the cell-guiding cementoinductive PLGA scaffolds was performed in the same way as described above.

The incorporation of growth and differentiation factors in cell-guiding PLLA scaffolds was performed in the same manner as with PLGA scaffolds.

2) Growth Factors Incorporation into Natural Biopolymer Cell-Guiding Scaffolds

Fibrin cell-guiding scaffolds were removed from the PBS and dried in a vacuum chamber at room temperature for 12 hours. The growth factors combination and concentration selected for rendering the cell-guiding scaffolds fibroinductive, osteoinductive, cementoinductive and angiogenic properties were exactly as those applied for the synthetic polymer scaffolds. The growth and differentiation factor mix was combined with 8 IU/ml of heparin solution and applied to the dry fibrin scaffolds inside the 12-well cell culture plates. Both cell-attachment facilitating ECM protein-containing (fibronectin and laminin combination) and unmodified fibrin scaffolds were kept in room temperature following the 50 μl heparin and growth factors-containing mix solution application for 30 minutes. The growth and differentiation factors-adsorbed fibrin cell-guiding scaffolds were then transferred to a freeze-dryer (Christ) and were freeze-dried at −20° C. under vacuum (pressure≦0.5 mmHg) for 10 days.

In both PLGA and fibrin cell-guiding scaffolds rendered with fibroinductive and angiogenic properties by the selection of the growth and differentiation factors combination described for that purpose, the concentration gradient of growth and differentiation factors was obtained along the lengths of the scaffolds in some specimens. For that purpose, growth factors of the said combination (bFGF, aFGF, IGF-I, PDGFββ, and VEGF) were reconstituted at 2× concentration (bFGF (200 μg/ml), aFGF (40 μg/ml), IGF-I (100 μg/ml), PDGFββ (100 μg/ml), and VEGF (200 μg/ml) and mixed together adding 50 μl from every growth factor solution into a vial. Following the thorough mixing of the growth factors by vortex, four different concentrations were prepared by combination of 100 μl of growth factor mix with 100 μl, 200 μl, 300 μl and 700 μl of heparin (8 IU/ml) containing solution. Thus, the concentrations of 2-fold, 1.5-fold, 1-fold and 0.5-fold of the used concentrations for the growth factors combination for fibroinductive and angiogenic cell-guiding scaffolds were obtained. The cell-guiding scaffolds were placed on a 35 mm plastic cell culture dish and were dented with a blade from the side borders at every 2.5 millimeter measured with reference grid paper. To achieve the concentration gradient along the length of the scaffolds, 20 μl of every concentration was applied with a micropipette transversely within the determined 2.5 mm regions. Following drying step for 15 minutes at vacuum chamber in room temperature, the next area was treated with the following growth factor concentration solution, thereby achieving four different areas of intermingling concentration gradients. Upon the completion of growth factors incorporation, both types (PLGA and fibrin) of the cell-guiding scaffolds were freeze-dried at −20° C. (pressure≦0.5 mmHg) for 10 days in the freeze-dryer (Christ) to stabilize the incorporated growth factors content.

### Example 2

E. Cell Proliferation and Migration Experiments with Human Periodontal Ligament Fibroblastic Cells (hPDLF) Inside the Cell-Guiding Fibrogenic and Angiogenic Scaffolds in Vitro.
1) Cell Culture and Expansion of hPDLF Cells

Human periodontal ligament fibroblastic cells were isolated and culture expanded as described (Inanc et al., Tissue Eng. 2006; 12(2): 257-66, and Inanc et al., J Biomed Mater Res A. 2007; 82(4): 917-26). Briefly, the periodontal ligament tissue from the middle third of the root of premolar teeth extracted due to orthodontic treatment needs was aseptically scraped with sterile blades and transferred to cell culture medium consisting of Dulbecco's Modified Eagles Medium (DMEM) supplemented with 15% Fetal Bovine Serum (FBS), 1% Non-Essential Amino Acid (NEAA) stock solution, 2 mM L-Glutamine, and 10× antibiotic stock solution (1000 U/ml penicillin and 500 μg/ml streptomycin) (all from Invitrogen). The periodontal ligament tissue was minced finely with the blades on 35 mm cell culture dish into pieces ˜1 mm³ and then transferred to 25 cm² polystyrene tissue culture flasks (Corning) containing 5 ml cell culture medium (DMEM containing 15% FBS, 1% NEAA, 2 mM L-Glut, 1% Pen/Strep). The explants culture was maintained in a humidified atmosphere of 5% CO₂ at 37° C., with medium change every other day. The culture was continued 3-6 weeks until proliferating fibroblasts became confluent and cells were enzymatically dissociated by % 0.05 trypsin/0.53 mM EDTA incubation for 5 minutes at 37° C. and passaged with a split ratio of 1:3. Subsequent expansion was performed in 75 cm² tissue culture flasks. The split ratio was kept at 1:3 and the cells usually reached confluence for 2-3 days at which point the next passage was performed. The cells from 4-6 passages were used in the experiments.

2) Characterization of hPDLF Cells

Human periodontal ligament fibroblastic cells' characteristic morphology of spindle shape was observed in a phase-contrast microscope (Nikon TS 100, Tokyo, Japan). Cells were controlled daily for their appearance, the state of expansion and confluence. Immunophenotypical characterization for the expression of cell surface antigens was performed with indirect immunohistochemistry as described (Inanc et al., Tissue Eng. 2006; 12(2):257-66). Briefly, hPDLF cell were seeded at 5×10⁴/cm² on 13 mm Thermanox™ cell culture treated coverslips (Nunc) in 24-well cell culture plate (Nunc) and cultured in an incubator at 37° C., 5% CO₂, and 90% humidity until reaching confluence. The cell-covered coverlips were washed 3 times with PBS at room temperature, fixed in an ice cold methanol for 5 minutes, and air dried. To block the endogenous peroxidase activity the samples were incubated for 5 minutes with peroxide block solution (LabVision, Fremont, Calif., USA), rinsed with PBS and then incubated for 5 minutes with Ultra V Block (Lab Vision) to block the non-specific binding. Primary antibodies used were mouse monoclonal anti-fibroblast surface antigen IgM (1:100, Sigma), goat polyclonal anti-collagen type I IgG (1:200, Santa Cruz Biotech., Santa Cruz, Calif., USA), and mouse monoclonal anti-fibronectin IgM (1:100, Sigma). They were reconstituted in PBS containing 1% bovine serum albumin (BSA, Sigma). Cell-covered coverslips were incubated with the primary antibodies overnight at 4° C., rinsed with PBS and incubated with species and isotype specific secondary antibodies for 30 minutes at room temperature. The secondary antibodies used were biotinylated rabbit anti-goat IgG and goat anti-mouse IgG (Zymed, San Francisco, Calif., USA). The enzymatic reaction was carried out with horseradish peroxidase (Lab Vision) and AEC chromogen (Lab Vision) was used to develop the red staining. The specimens were analyzed under inverted phase-contrast microscope (Nikon), and digital light microscope (Leica DM 4000 B, Wetzlar, Germany).

3) Viability and Proliferation Experiments of hPDLF Cells on Cell Culture Treated Surfaces

The cell viability in confluent hPDLF monolayer on tissue culture plastic was evaluated as described (Inanc et al., Tissue Eng. 2006; 12(2): 257-66). Following trypsinization of the cells, trypan blue (Sigma) dye exclusion assay based on staining of the dead cells was performed. The representative counts on hemocytometer under phase-contrast microscope consistently demonstrated the cellular viability of above 95%.

Cell proliferation kinetics were investigated using 5-bromo-2′-deoxyuridine (5-BrdU, Sigma) incorporation. hPDLF cells were seeded at ˜2×10⁴ cell/cm² on 22 mm Thermanox™ coverslips inside 6-well plate, and 16 ng/ml/day of BrdU was added to the culture medium. The specimens were fixed with ice-cold methanol at 24, 48 and 72 hours and following indirect immunohistochemical processing with anti-BrdU primary and then secondary antibody treatments, the BrdU cells were visualized with 3-3′-diaminobenzidine (DAB, Santa Cruz Biotech.) staining and cell count was performed in representative fields under phase-contrast microscope. The cell proliferation percent was determined as

$$\frac{proliferating cells}{non \text{-} proliferating cells} \times 100$$

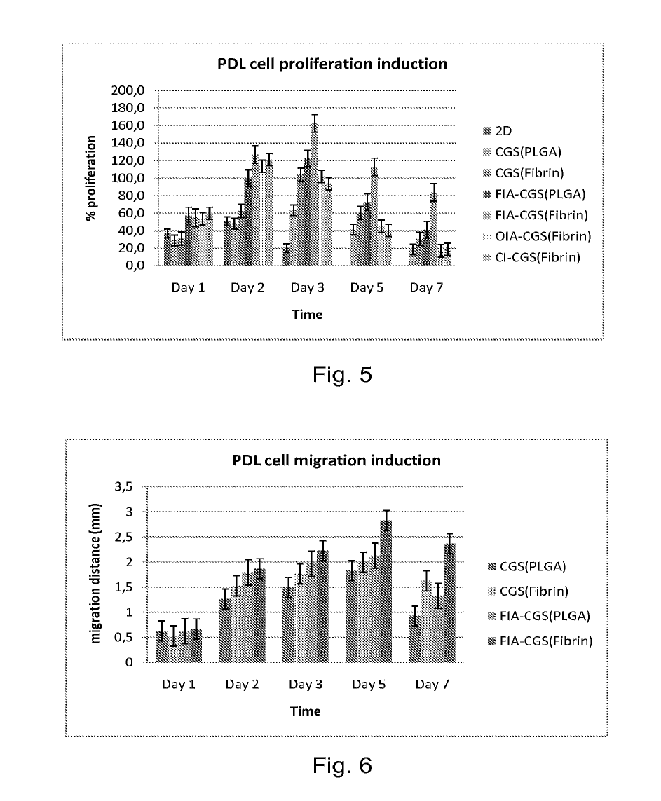

in a given field. The results obtained from five independent experiments are presented in FIG. 5.

The majority of the hPDLF cells cultured on 22 mm Thermanox™ coverslip surfaces adhered on the surface after the initial 2-3 hours following the seeding, and elicited fibroblastic morphology at 24 hours. Cell proliferation based on the counting of BrdU⁺ nuclei was found to be %37±4.69 for the first day. The percent increased (%50.8±4.66) for the second day and again decreased (%26.2±6.18) at the third day. At 72 hours, cells completely populated the coverslip area, reaching confluent state.

4) Viability, Proliferation and Migration Experiments of hPDLF Cells Inside Cell-Guiding Scaffolds without Cell Attachment Enhancing Modification and Growth Factors Incorporation

The cytocompatibility of the cell-guiding scaffolds fabricated from either PLGA or fibrin were measured using MTT (3-(4,5-dimethyl-2-thiasolyl)-2,5-diphenyl-2H-tetrazolium bromide)-based assay evaluating the mitochondrial dehydrogenase activity characteristic for the living cells, as described (Inanc et al., Tissue Eng. 2006; 12(2): 257-66). The mitochondrial dehydrogenase converts the tetrazolium salt into formazan crystals and the dissolved formazan was then read spectrophotometrically at 570 nm wavelength.

Human periodontal ligament fibroblastic cells were trypsinized and resuspended in culture medium at a concentration of ˜5×10⁶ cells/ml. 20 μl of cell suspension was added dropwise on the surface of fibrin or PLGA scaffolds of ˜10×5×1.5 mm dimensions placed in a wells of 6-well cell culture plate. 1.5 ml of culture medium was added around the scaffolds without disturbing the cells and the cell-seeded scaffolds were placed in an incubator at 37° C. and 5% CO₂. After 4 hours, 2 ml of culture medium was added and the cell-containing scaffolds were cultured for 24, 48 and 72 hours. At the indicated time points, the scaffolds were transferred into new 6-well cell culture plates to eliminate cells on the plate surface and covered with 2 ml of MTT solution (5 mg/ml) in DMEM supplemented with 0.5% FBS and 1% Pen/Strep solution. The cell-containing scaffolds were then incubated for 4 hours at 37° C. and 5% CO₂. Hundred microliters of aliquots were then taken from each well in triplicate and placed into a 96-well plate, which was read on a microplate reader at 570 nm (Varian, Calif., USA). To correlate absorbance with cell number, hPDLFs were cultured on 6-well cell culture plates, assayed with MTT as described and then trypsinized and counted using hemocytometer. The results from MTT experiments demonstrated that hPDLFs viability remained above 90% for all tested time points (24 h, 48 h, and 72 h) in both PLGA and fibrin scaffolds. While the absorbance levels from fibrin cell guiding scaffolds were higher than the PLGA scaffolds at 48 and 72 hours, the difference can be attributed to the higher cell numbers as a result of higher proliferative rates inside these scaffolds compared to the PLGA. This difference becomes more pronounced with the culture time, as can also be seen from the results of the cell proliferation experiments (FIG. 5).

To assay the hPDLF cell proliferation kinetics inside the unmodified cell-guiding fibrin or PLGA scaffolds, trypsinized human periodontal ligament fibroblastic cells were seeded at ˜1×10⁶ cells/scaffold. Seeded scaffolds were cultured in 6-well cell culture plates in 2 ml/well cell culture medium. To label the newly synthesized DNA and thus detect the proliferating cells, the medium was supplemented with BrdU (16 ng/ml/day) for 24 hours at indicated time points for 7 days as described with some modifications (Sales et al. Circulation 2006; 114 (1 Suppl):1193-9). The cell-containing scaffolds were formalin-fixed and paraffin-embedded at days 1, 2, 3, 5, and 7. Representative sections of ˜5 μm were obtained, processed for indirect immunohistochemistry with anti-BrdU primary and then secondary antibody (Santa Cruz Biotech.) treatment, and BrdU⁺ cells were visualized with DAB staining, and counterstained with hematoxylin (Sigma). The number of BrdU⁺ cells and total number of cells were counted in six randomly selected areas from each representative section under inverted light microscope at 400×. For any given field, the cell proliferation percent for the predetermined time points was calculated as

$$\frac{proliferating cells}{non \text{-} proliferating cells} \times 100.$$

At least three different sections from every scaffold were analyzed and the cell proliferation percent inside the scaffolds was determined as the mean value from six counts/section, and three sections/scaffold. The data from five independent experiments is presented in FIG. 5.

Results indicate that proliferation kinetics of hPDLF cells are significantly influenced by the biomaterial types of the scaffolds (PLGA or fibrin), the growth factors incorporation into the scaffolds with cell attachment enhancing modifications, and the combination of growth factors used. Although cell proliferation percent was similar at Day 1 between the cell-guiding PLGA [CGS(PLGA)] and fibrin[CGS(Fibrin)] scaffolds that does not contain growth factors and also between the four types of scaffolds with incorporated growth factor combinations (cell-guiding fibroinductive and angiogenic PLGA scaffolds[FIA-CGS(PLGA)], cell-guiding fibroiductive and angiogenic fibrin scaffolds[FIA-CGS(Fibrin)], cell-guiding osteoinductive and angiogenic fibrin scaffolds[OIA-CGS(Fibrin)], and cell-guiding cementoinductive fibrin scaffolds[CI-CGS(Fibrin)]), the proliferation rates inside the growth factors-containing scaffolds were up to two-fold grater than those not contain growth factors. The proliferation rate was also higher in 2D compared to the latter group at that time point, however single cell layer experiments were performed as a reference and not for the purpose of directly comparing the proliferation rate between 2D and 3D, where different extracellular environment can be expected to influence the results in a multifaceted way, making the comparison between two cell culture types inadequate. At Day 2 the proliferation rate increased approximately 1.5-fold for CGS(PLGA) and FIA-CGS(PLGA) (%29±6.78 vs. %48.2±4.02, and %57.2±8.87 vs. %100±9.46 respectively), while approximately 2-fold increase was observed for all types of fibrin scaffolds (CGS(Fibrin)—%31±4.69 vs. %62.6±4.67; FIA-CGS(Fibrin)—%55±6.6 vs. %127±8.43; OIA-CGS(Fibrin)—%53.67±6.65 vs. %113.6±7.67; CI-CGS(Fibrin)—%59.8±5.97 vs. %121.2±10.6 for Day 1 and Day 2 respectively). The highest proliferation rate was associated with FIA-CGS(Fibrin) with %127±8.43, followed by CI-CGS(Fibrin) with %121.2±10.6, whilst the lowest was in CGS(PLGA) with %48.2±4.02. The trend of increase continued upward at Day 3 for scaffolds not containing growth factors and also for the cell-guiding fibroinductive and angiogenic scaffolds. However, it slightly diminished in OIA-CGS(Fibrin) and more prominently in CI-CGS(Fibrin). Thus, the proliferation rates for that time point were as follows: CGS(PLGA)-%63.4±7.13; CGS(Fibrin)-%104±11.9; FIA-CGS(PLGA)-%122.4±7.92; FIA-CGS(Fibrin)-%162.6±15.3; OIA-CGS(Fibrin)-%102±8.63; and CI-CGS(Fibrin)-%93.6±7.4. At the fifth day of experiments, cell proliferation percent was down in all groups from the levels at the third day. The most notable decrease has been observed in osteoinductive and cementoinductive cell-guiding scaffolds with more than fifty percent diminished cell proliferation in both groups. One third decrease was also noted in PLGA and fibrin cell-guiding scaffolds not containing growth factors, with PLGA scaffold levels similar to the OIA-CGS and CI-CGS groups. The cell proliferation levels in fibroinductive and angiogenic cell-guiding scaffolds were ˜2/5 down from the third day levels. Thus, at fifth day the proliferation percent of the hPDLF cells in FIA-CGS(Fibrin) was nearly three times greater than the CGS(PLGA), OIA-CGS(Fibrin) and CI-CGS(Fibrin). The percent cell proliferation for every group was as follows: CGS(PLGA)-%41.4±6.07; CGS(Fibrin)-%60.4±7.4; FIA-CGS(PLGA)-%72.8±10.9; FIA-CGS(Fibrin)-%112.8±12; OIA-CGS(Fibrin)-%45.2±8.7; and CI-CGS(Fibrin)-%40.2±8.07. At the seventh day, the cell proliferation continued to decrease in all groups (CGS(PLGA)-%18.8±3.96.51; CGS(Fibrin)-%30.6±4.04; FIA-CGS(PLGA)-%41±5.57; FIA-CGS(Fibrin)-%83.8±7.01; OIA-CGS(Fibrin)-%17.2±4.76; and CI-CGS(Fibrin)-%18.8±4.66), with the highest level in FIA-CGS(Fibrin), being 4-fold higher than CGS(PLGA), OIA-CGS(Fibrin), and CI-CGS(Fibrin) groups, ˜3-fold higher than CGS(Fibrin), and 2-fold higher than FIA-CGS(PLGA).

Overall data indicate similar pattern of proliferation kinetics in the groups, where hPDLF cells increase their proliferation rate up to third day, which decreases thereafter. Both scaffold biomaterial types and growth factors' presence and composition affects the rates of proliferation, where fibrin structure and fibroinductive and angiogenic growth factor composition appears to stimulate the hPDLF proliferation better than PLGA does. Osteoinductive and angiogenic as well as cementoinductive cell-guiding scaffolds elicit a reduced cellular proliferation notably after Day 3, and that reaches the levels similar to cell guiding PLGA scaffolds not containing growth factors at Day 7. Human periodontal ligament fibroblastic cell proliferation is enhanced in a highest degree by fibroinductive and angiogenic cell-guiding fibrin scaffolds, followed by fibroinductive and angiogenic cell-guiding PLGA scaffolds, and in a lowest degree by cell-guiding PLGA scaffolds. Cell-guiding fibrin scaffolds not containing growth factors and OIA-CGS(Fibrin) and CI-CGS(Fibrin) act differently on cell proliferation induction, where the latter induce greater amount of cell proliferation at the first two days, attain similar levels at the third day and subsequently diminish that rate compared to the former scaffolds. The osteoinductive and cementoinductive scaffolds composition of growth factors seems to suppress the hPDLF cell proliferation at certain time point, probably as a result from the actions of BMP's, known to induce cell differentiation whilst suppressing proliferation of various cells, as demonstrated in scientific literature.

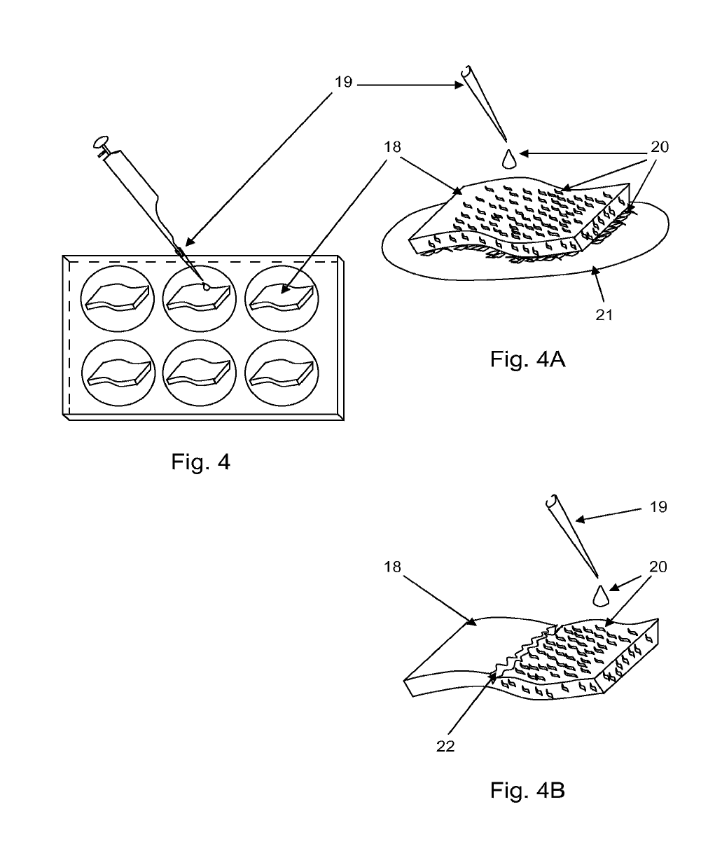

The effects of unmodified fibrin and PLGA cell-guiding scaffolds on the migration of hPDLF cells in vitro were determined along the thickness or length of the scaffolds (FIG. 4). For the transverse migration experiments, the scaffolds were placed in 6-well culture plates and were seeded with ˜3.5×10⁶/cell/scaffold and cultured for 4 hours at 37° C. in 5% CO₂ (FIG. 4A). The cell seeded scaffolds were then transferred to new 6-well plates on 22 mm Thermanox™ coverslips and cultured up to 5 days, with a medium change every other day. At days 1, 2, 3, and 5 the cell-containing scaffolds were fixed with formalin and embedded in paraffin, and ˜5 μm thick transverse sections were obtained. The specimens were stained with hematoxylin and eosin (H&E), and staining of cell nuclei was performed with Hoechst 33256 (2′-(4-Hydroxyphenyl)-5-(4-methyl-1-piperazinyl)-2,5′-bi(1H-benzimidazole)trihydrochloride, Sigma). Cell distribution was analyzed under phase-contrast microscope with fluorescent attachment (λₑₓ: 346 nm, λₑₘ: 460 nm), and also inverted light microscope at 200×. The coverslips were fixed at the same time points with ice-cold methanol and stained with H&E, and then cell density on the coverslip surface was observed under inverted light microscope (200×). Histological specimens indicated that the PDLF cells were distributed throughout the 1.5 mm thickness of both PLGA and fibrin scaffolds at the end of Day 1, with the majority of the cells locating near the upper surface. At Day 2, the cell density inside the scaffolds increased and the whole thickness was densely populated at the end of Day 3. Cells were also present on the coverslip surface at Day 3 and reached semi-confluence at the end of Day 5 of culture.

For longitudinal migration experiments hPDLF cells were seeded on a half or quarter of the 20 mm-long scaffolds. The scaffolds were dissected with a sterile blade so as to incompletely separate them from the middle line perpendicular to the length of the scaffold or separate the quarter of it in a similar manner. The separation line was not extended to the bottom of the 1.5 mm-thick scaffold, but was sufficient to bend the two parts apart (FIG. 4B). Then, the cell suspension was seeded at a single step on one half or quarter of the scaffold with ˜10 mm or ˜5 mm length. Approximately 3.5×10⁶ and 7×10⁶ cells were seeded to the scaffold parts depending on the size (5 mm or 10 mm length respectively). The bent scaffold parts were kept as such for 2 hours in 3 ml cell culture medium inside 6-well culture plates to allow for the initial cell attachment. Then, the scaffolds were carefully removed from the plates and transferred into new cell culture plates with two semi-separated parts united in a close contact again. The reunited cell-containing and unseeded parts of the scaffolds were cultured together in cell culture medium up to 7 days. At days 1, 2, 3, 5, and 7 the scaffolds were fixed with formalin and embedded in paraffin, and three serial ˜5 μm thick longitudinal sections along the length of unseeded scaffold portion were obtained. The specimens were stained with H&A, and staining of cell nuclei was performed with Hoechst 33256. Analysis was performed for the presence and distribution of cells under inverted light microscope and fluorescent phase contrast microscope at 200×. The measurements were performed against a translucent reference grid (0.1 mm sensitivity) juxtaposed to the microscope slide and expressed as millimeters. The data from in vitro cell migration experiments in unmodified fibrin and PLGA scaffolds (mean±SD from three experiments) is presented in FIG. 6.

5) Viability, Proliferation and Migration Experiments of hPDLF Cells Inside Cell-Guiding Scaffolds Following Cell Attachment Enhancing Modification and Growth Factors Incorporation

Cytocompatibility, and effects on hPDLF cell proliferation and migration of PLGA and fibrin cell-guiding scaffolds following cell attachment enhancing modification and growth factors incorporation were also determined with methods described for the unmodified scaffolds. Thus, MTT was used to determine the cell viability upon cell seeding for 24, 48 and 72 hours, cell proliferation kinetics were measured using BrdU⁺ incorporation method for 1, 2, 3, 5, and 7 days, and cell migration experiments along the scaffolds thickness and length were performed for 1, 2, 3, 5, and 7 days. The data from cell proliferation experiments is presented in FIG. 5, and cell migration experiments data along the scaffold lengths is presented in FIG. 6.

The results from the cell migration experiments demonstrated the ability of four types of cell-guiding scaffolds to support the migration of hPDLFs in a given direction in vitro, in this instance the longitudinal axis of the unpopulated scaffold parts. The front line of the migration extent was considered as the area on a histological specimen demonstrating presence of multiple cells adjacent to an area of empty scaffold matrix. The individual cells not being able to be associated with the multiple cell-containing areas were left outside of consideration. Reference translucent grid with scale of 0.1 mm was used to determine the distance from the migrating front line of hPDLFs from the semi-separation line of the scaffold, from where the migration began. The cell migration distance for every time point was determined by subtracting the mean amount of migration distance from the previous time point for a given group from the total amount of migration distance at the assayed time point. Thus, the mean cell migration distance for the indicated time was determined for every group of cell-guiding scaffolds. Unmodified cell-guiding PLGA, unmodified cell-guiding fibrin, cell-guiding fibroinductive and angiogenic PLGA, and cell-guiding fibroinductive and angiogenic fibrin scaffold were the four types of cell-guiding scaffolds from which the results for hPDLF cell migration were obtained.

There were not statistically significant differences between groups at Day 1 (CGS(PLGA)-0.63±0.21 mm; CGS(Fibrin)-0.53±0.15 mm; FIA-CGS(PLGA)-0.63±0.25 mm; FIA-CGS(Fibrin)-0.67±0.15 mm). At Day 2 the lowest migration rate was seen in CGS(PLGA) with 1.27±0.15 mm, and the highest in FIA-CGS(Fibrin) with 1.87±0.15 mm. However, the results from different biomaterial scaffolds were compared against each other at every measured time point for statistical significance with Student t tests. Thus, at Day 2 the cell migration distance in CGS(PLGA) (1.27±0.15 mm) were not significantly different from the CGS(Fibrin) (1.53±0.21 mm), which was the case also for FIA-CGS(PLGA) (1.8±0.17 mm) vs. FIA-CGS(Fibrin) (1.87±0.15 mm). At that time point the growth factors composition significantly affected the migration amount of hPDLFs only in PLGA scaffolds. At the third day, the growth factors composition elicited barely significant difference in PLGA scaffolds (CGS(PLGA)-1.5±0.17 mm vs. FIA-CGS(PLGA)-1.97±0.23 mm, p=0.049). However, the migration amount was nearly 1.5-fold greater in cell-guiding fibroinductive and anigiogenic fibrin scaffolds (2.23±0.21 mm) compared to the unmodified cell-guiding PLGA scaffolds (1.5±0.17 mm). The results between growth factors-containing PLGA (1.97±0.23 mm) and fibrin (2.23±0.21 mm) scaffolds were not significantly different.

The next time point of measurement was the 5^{th} day, accounting for a two day migration time. Here, the fibrin cell-guiding scaffolds appeared to sustain better cell migration induction compared to the matched PLGA scaffolds. The lowest level was in CGS(PLGA) with 1.83±0.21 mm, followed by CGS(Fibrin) with 2.0±0.17 mm, with similar results for FIA-CGS(PLGA) (2.13±0.31 mm), and the highest rate was measured in FIA-CGS(Fibrin) with 2.83±0.15 mm. However, when groups were compared, the difference was significant between fibrin scaffolds, but not the PLGA ones. At the last measurement point, the 7^{th} day results indicate overall decrease in hPDLF cell migration inside all of the cell-guiding scaffold types, most notably in unmodified PLGA (0.93±0.15 mm), followed by growth factors-containing fibroinductive and angiogenic PLGA (1.33±0.21 mm), unmodified fibrin (1.63±0.25 mm) and growth factors-containing fibroinductive and angiogenic fibrin (2.37±0.25 mm) scaffolds. The differences were not significant only between CGS(PLGA) and FIA-CGS(PLGA).

The results indicate that cell-guiding fibroinductive and angiogenic fibrin scaffolds are the best cell-guiding scaffold type with regard to hPDLF cell migration in vitro, where compounded mean values reach 9.97±0.91 mm, or cover nearly the entire length of a 10 mm-long scaffold structure. In cell-guiding fibroinductive and angiogenic PLGA scaffolds the compounded mean values±SD were 7.86±1.17 mm, for unmodified cell-guiding fibrin scaffolds they were 7.46±0.99 mm, and for unmodified cell-guiding PLGA scaffolds the rate was 6.16±0.89 mm. Thus, it can be concluded that FIA-CGS(Fibrin) induce hPDLF migration along the whole length of a 10 mm scaffold; FIA-CGS(PLGA) and CGS(Fibrin) induce it to the extent of 75-80%, and unmodified cell-guiding PLGA scaffolds achieve a cell migration conduction up to 60% of the whole scaffold length.

6) Extracellular Matrix Synthesis and Deposition by hPDLF Cells in Cell-Guiding Fibroinductive and Angiogenic Scaffolds In Vitro

Cell-guiding fibroinductive and angiogenic scaffolds' abilities to support cell functionality was evaluated in vitro with hPDLF cell seeding and culturing inside the growth factors-containing fibrin scaffolds without adsorbed cell attachment enhancing ECM proteins. hPDLF cells were trypsinized and seeded on scaffolds placed inside 6-well culture plates at ˜7×10⁶ cells/scaffold, and cultured for 4 hours with 1.5 ml cell culture medium at 37° C. in 5% CO₂. 1.5 ml cell culture medium was added to a total volume of 3 ml/well, and the cell-containing scaffolds were cultured for up to 21 days with a medium change every other day. At the 7^{th}, 14^{th}, and 21^{st} days, the scaffolds were removed from the plates, rinsed three times with sterile PBS, and then were formalin-fixed and paraffin embedded. ˜5 μm thick sections were obtained, fixed on microscope slides and deparafinized. The specimens were assayed for the deposition of collagen type I (Col I), collagen type III (Col III) and fibronectin with indirect immunohistochemistry. Following endogenous peroxidase quenching and blocking of non-specific antibody binding as described earlier, the specimens were incubated with primary antibodies against Col I (goat polyclonal anti-Col I IgG, 1:200, Santa Cruz Biotech.), Col III (mouse monoclonal anti-Col III IgM, 1:100, Sigma), and fibronectin (mouse monoclonal anti-fibronectin IgM, 1:100, Sigma) at 4° C. overnight, followed by biotinylated species and isotype specific secondary antibody (rabbit anti-goat IgG, 1:200, and goat anti-mouse IgG, 1:200, Zymed) incubation for 30 minutes at room temperature. Enzymatic reaction was carried out with streptavidin horseradish peroxidase, followed by staining with AEC chromogen to develop red staining. Specimens were slightly counterstained with hematoxylin and analyzed under digital light microscope (Leica DM 4000 B). The immunohistomorphometric evaluation of the staining percent was performed as described (Inanc et al., J Biomed Mater Res A. 2007; 82(4): 917-26). Briefly, six randomly selected areas were evaluated from each specimen for positively stained areas as determined with analytical software (Leica QWin Plus, Wetzlar, Germany). The percent of positively stained area to the total area of any given field was the staining percent of a given ECM protein, and was expressed as the mean value of six measurements. The data from three independent experiments on staining percent for the selected markers is presented in Table-1.

Collagen type I is the principal extracellular matrix protein in periodontal ligament and constitutes up to %95 of all the collagen of periodontal ligament fibers. Thus, it is also the main collagen type synthesized and deposited by human periodontal ligament fibroblastic cells in culture, and at 7^{th} day the staining level in the cell-guiding fibroinductive and angiogenic fibrin scaffold specimens was %72±6.56. At the same time point, the staining for collagen type III, which is formed predominantly at the early phases of wound healing was %18.7±3.06. As another important extracellular matrix molecule, the fibronectin was detected at %11.33±3.51. At the end of the second week, Col I level increased to %83.33±6.11. However the difference between the two time points was not statistically significant. Notably, the Col III level decreased to %4.33±2.08 suggesting that some remodeling activity might take place inside the cell-containing scaffolds. The fibronectin remained at comparable levels with %15.67±4.51. At the end of the third week, Col I staining percent further increased to %92±4.58, Col III staining was not detectable at measurable levels against the background staining, and fibronectin staining remained at similar levels compared to the previous time points with %13±2.65.

7) Osteogenic Differentiation Induction of hPDLF Cells in Cell-Guiding Osteoinductive and Angiogenic Scaffolds In Vitro

The cell-guiding osteoinductive and angiogenic fibrin scaffolds containing growth and differentiation factors combination determined for osteoinductive scaffolds (bFGF, IGF-I, PDGFββ, BMP-2, BMP-4, BMP-7, BDNF, and VEGF) were seeded with trypsinized hPDLF cells (˜7×10⁶ cells/scaffold) and cultured in 6-well culture plates for 24 hours in a standard cell culture medium. Then, medium was changed with osteoinductive medium (OIM), the medium used for hPDLF cell expansion supplemented with 10 mM β-glycerophosphate, 50 μg/ml ascorbic acid and 10 nM dexamethasone. The osteogenic differentiation culture was continued for 28 days with the medium change every other day. Cell-containing scaffolds were fixed with 10% neutral buffered formalin overnight at 4° C. After paraffin embedding and sectioning, the ˜5 μm-thick sections were processed for immunohistochemistry. Primary antibodies used were: goat polyclonal anti-osteopontin IgG (1:100, Santa Cruz Biotech.), rabbit polyclonal anti-bone sialoprotein IgG (1:200, Alexis Biochemicals, Lausen, Switzerland), and goat polyclonal anti-osteocalcin (1:100, Santa Cruz Biotech.). Specimens were incubated with primary antibodies overnight at 4° C., followed by species and isotype specific biotinilated secondary antibody (mouse anti-goat and goat anti-rabbit, Zymed) incubation for 30 minutes at room temperature. Enzymatic reaction was carried out with streptavidin horseradish peroxidase (Lab Vision), and the AEC chromogen (Lab Vision) was used according to manufacturer's instructions to develop 1315 red staining. The staining for the selected markers was visualized under inverted digital microscope and the percent of staining for every marker was determined by immunohistomorphometry. The data from osteogenic induction experiments is presented in Table-1.

The capacity of human periodontal ligament fibroblastic cells for osteogenic induction to some extent under the influence of osteogenic medium in vitro was previously demonstrated in cell-seeded PLGA scaffolds (Inanc et al., Tissue Eng. 2006; 12(2): 257-66.) and also chitosan/hydroxyapatite (Inanc et al., J Biomed Mater Res A. 2007; 82(4): 917-26.) microsphere encapsulated cells. Four week-long osteogenic induction of hPDLFs seeded in cell-guiding osteoinductive and angiogenic fibrin scaffolds resulted with the synthesis and deposition of bone specific extracellular matrix molecules osteopontin, bone sialoprotein and osteocalcin at levels of %22±6.9, % 26±4.8, and %32±7.2 respectively.

TABLE 1
Fibrogenic and osteogenic ECM protein expression (%)

| | Fibrogenic markers in FIA-CGS(fibrin) | | | Osteogenic markers in OIA-CGS(fibrin) | | |
|---|---|---|---|---|---|---|
| Days/Markers | Col I | Col III | Fibronectin | OSP | BSP | OSC |
| 7 | 72 ± 6.56^{¶} | 18.7 ± 3.06* | 11.3 ± 3.51 | — | — | — |
| 14 | 83.3 ± 6.11 | 4.3 ± 2.08* | 15.7 ± 4.51 | — | — | — |
| 21 | 92 ± 4.58^{¶} | NA | 13 ± 2.65 | — | — | — |
| 28 | — | — | — | 19 ± 4.36 | 25.3 ± 4.16 | 32.7 ± 5.03 |

Abbreviations: Col I: Collagen Type I; Col III: Collagen Type III; OSP: Osteopontin; BSP: Bone Sialoprotein; OSC: Osteocalcin; FIA-CGS(fibrin): cell-guiding fibroinductive and angiogenic fibrin scaffolds; OIA-CGS(fibrin): cell-guiding osteoinductive and angiogenic fibrin scaffolds.; ECM: Extracellular matrix. Data represent mean (%) ± SD from three experiments; Statistical significance set at p < 0.05.
^{¶}p < 0.05;
*p < 0.01

### Example 3

F. Periodontal Ligament Tissue Regeneration Following Implantation of Cell-Guiding Fibrogenic and Angiogenic Scaffolds into the Experimental Periodontal Defects in Dogs.

### 1) Formation of Experimental Periodontal Defects in Dog Premolar Teeth

Six male mongrel dogs (weighing 8-10 kg each) were used in periodontal regeneration procedures in experimental periodontitis defects using cell-guiding fibroinductive and angiogenic, osteoinductive and angiogenic, and cementoinductive fibrin scaffolds of the invention. The surgical procedures were performed under general anesthesia with intravenous injection of Pentobarbital sodium administration (25-30 mg/kg). Mucoperiosteal flaps were raised on third and fourth premolars on both sides of the mandible and alveolar bone from the buccal root surface of the third and fourth premolars was removed initially with slow-speed handpiece and carbide burr under physiologic saline irrigation, and then mechanically with bone chisels. Orthodontic ligature wires were placed at cementoenamel junction (CEJ) level, and the flaps were sutured over the defects with interrupted 3/0 silk sutures. The dogs were fed with a soft diet for 4-week period during which the denuded root surfaces were covered with microbial dental plaque, developing experimental periodontal disease on the cementum surfaces. The ligature wires were removed at the end of the 4^{th} week and teeth surfaces were cleaned with mechanical debridement. The experimental periodontitis areas around 3^{rd} and 4^{th} premolar teeth were cleaned daily with a brush and rinsed with 0.5% Clorhexidine gluconate for 1 week to treat the gingival inflammation in the area.

2) Surgical Treatment of the Dogs' Premolar Teeth Periodontal Defects with Cell-Guiding Fibroinductive and Angiogenic, Osteoinductive and Angiogenic and Cementoinductive Scaffolds

The experimental regenerative procedures were performed at the end of the 5^{th} week under general anesthesia with IV-administered Pentobarbital sodium and local anesthesia with Lidocaine HCl. The mucoperiosteal flaps were removed again on the 3^{rd} and 4^{th} premolars, the planning with manual periodontal instruments was performed on the diseased root surfaces, and a horizontal notch was dented on the most apical part of the exposed root surfaces. Then, the buccal root surfaces of the mesial and distal roots of the 3^{rd} and 4^{th} premolar teeth were covered with cell-guiding fibroinductive and angiogenic, or osteoinductive and angiogenic, or cementoinductive scaffolds of the present invention. The symmetrical tooth root surfaces were treated with an expanded polytetrafluoroethylene(ePTFE) membranes, preventing the epithelial and gingival fibroblastic cell migration into defect area according to the guided tissue regeneration (GTR) principle. Fibroinductive and angiogenic, osteoinductive and angiogenic, or cementoinductive cell-guiding fibrin scaffolds of approximately 5.5×5×1.5 mm dimensions were fixed on the prepared root surfaces using the fibrin glue on the borders of the scaffolds. ePTFE membranes were placed and sutured on the top of the treated area at a level slightly above the CEJ in order to exclude the downward migration of epithelial and gingival fibroblastic cells. The flaps were elongated with periosteal fenestration and sutured in a coronal position with interrupted silk sutures. The dogs were fed with a soft diet during the initial 4 weeks after the treatment and plaque control was performed with daily brushing and rinsing with 0.5% Clorhexidine gluconate until suture removal. The sutures were removed after 2 weeks.

### 3) Histological and Histomorphometric Evaluation of the Regenerated Tissue Formation

At the end of 4 and 12 weeks after the cell-guiding scaffold implantation procedures, the dogs were anesthetized again with IV Pentobarbital sodium and local anesthesia with Lidocaine HCl. Following vertical incision with blades on interdental gingiva, vertical separation was performed with thin rotating separator discs under saline irrigation through the interproximal alveolar bone on the mesial and distal portions of the 3^{rd} and 4^{th} premolar teeth. The third and fourth premolar teeth on both sides of the mandible were removed together with the buccal and lingual bone and related periodontal structures, and the animals were sacrificed with the overdose of Pentobarbital sodium. The premolar teeth with the surrounding periodontal tissues were fixed in 10% neutral buffered formalin for 24 hours, and decalcified in trifluoroacetic acid (3%) and EDTA (18%) for 15 days, sectioned to 2-mm thick slices along the bucco-lingual plane using 1385 diamond separator discs. The slices were again demineralized in 18% EDTA for one week, dehydrated, embedded in paraffin, and serial slices of 5 μm thickness were obtained by microtome, placed on gelatin-coated glass slides, and stained with H&E or 1% Toluidine blue. 5 slices obtained 100 μm apart from the mid-buccal part of each root were used for the histometric measurements performed under inverted light microscope at 20×. The following areas were measured as linear mm distances from 5 specimens for each root: (i) apical extension of root planning (aRP, dented notch) to the cementoenamel junction (CEJ); (ii) aRP to coronal level of new cementum (cNC); (iii) aRP to coronal level of new alveolar bone (cNB); (iv) CEJ to apical extension of epithelial migration; (v) aRP to coronal level of connective tissue. The newly formed connective tissue was evaluated as percent of connective tissue length with varying fiber orientation and inflammatory cell infiltration (% NCT) and percent of periodontal ligament tissue with functional (perpendicular to oblique) fiber orientation (% NPDL(f)). The measurements were performed against a reference grid (0.1 mm) juxtaposed to the microscope slide. Statistical analysis was performed with Student t test for pairwise comparisons, and one-way ANOVA for multiple comparisons. The level of significance was set at p<0.05.

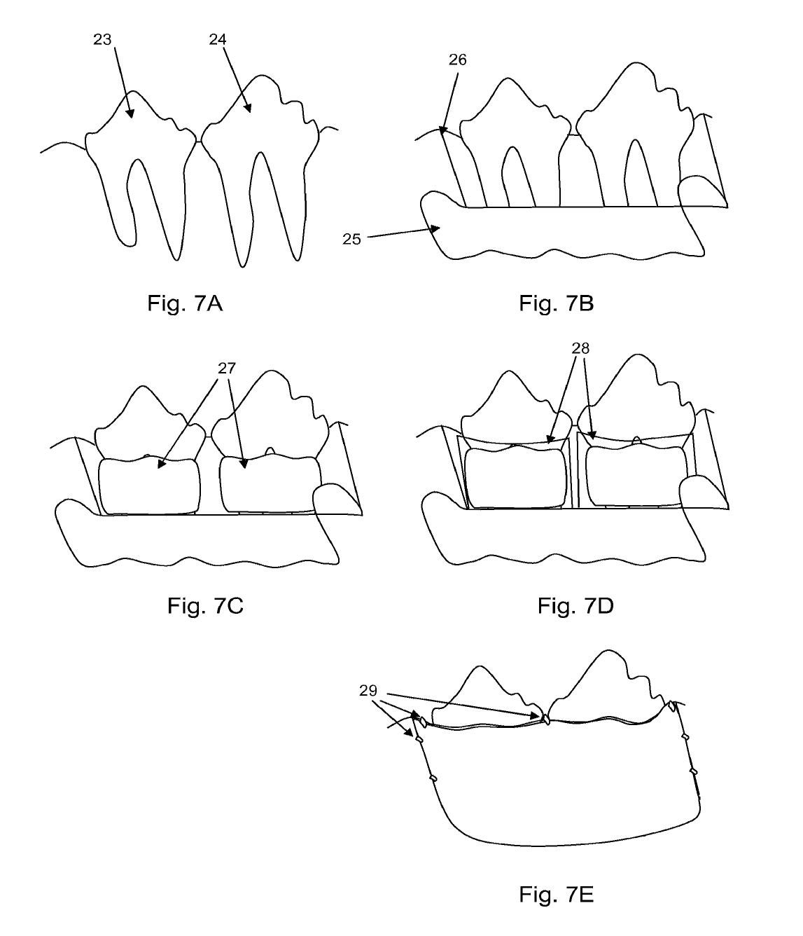

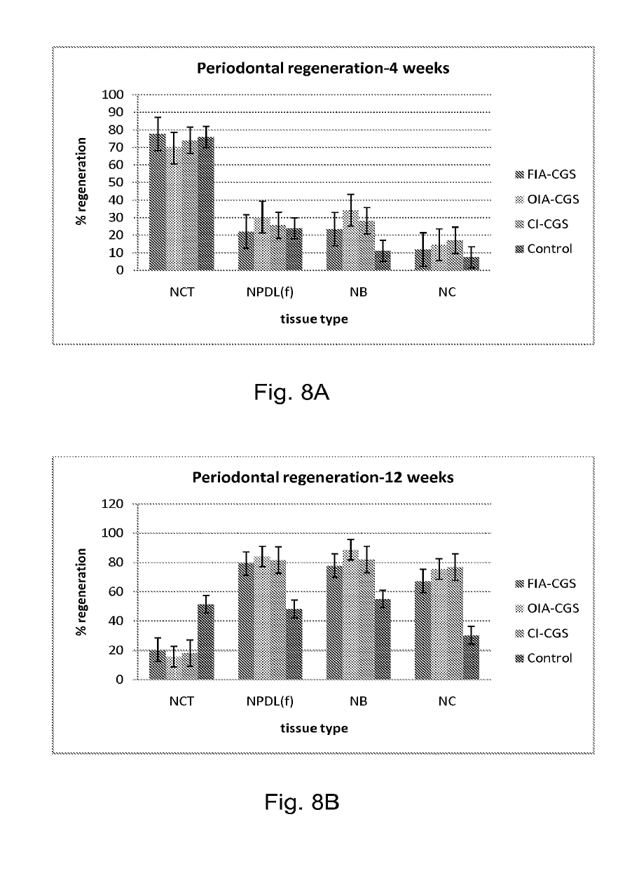

The treatment procedures of dog mandibular premolar teeth periodontal defects with cell-guiding scaffolds were schematically illustrated in FIG. 7. The results of the histometric measurements are presented as mean±SD obtained from 5 different slices for each root of a premolar, and total of 20 slices from every condition and time point, and are presented in FIG. 8.

Since all the used cell-guiding scaffolds in experiments were made of fibrin, only the incorporated growth factor content's effects and different cell-guiding scaffolds' effects on periodontal regeneration versus guided tissue regeneration procedure (barrier membrane only containing group) were compared. At 4 weeks, newly formed connective tissue covered majority of the distance between the reference notch on the tooth root surfaces and the most coronal measurement reference, the cemento-enamel junction in all groups. Of that, up to 78% was new connective tissue with random fiber orientation and inflammatory cell infiltration, while the periodontal ligament with functional fiber orientation remained between 22-30% depending on the group, predominantly located in the apical parts of the defect area. The difference between the groups as well as controls with regard of these two parameters was not statistically significant for the 4^{th} week time point. Both for 4^{th} and 12^{th} week measurements % NCT and % NPDL(f) were complementary, i.e. the cumulative of the two corresponding values are equal to %100, the total length of connective tissue formed along the root surface area in the defect site. While the results for the newly formed periodontal ligament tissue were similar between the experimental and control groups at Week 4, there was notable difference at Week 12. The % NPDL(f) increased to %79.4±8.71, %84.2±6.98, and %81.8±9.28 for FIA-CGS, OIA-CGS, and CI-CGS respectively, while it remained at %48.4±4.39 in the control specimens. Thus, it can be concluded from the results that cell-guiding fibroinductive and angiogenic, osteoinductive and angiogenic and cementoinductive scaffolds induce the regeneration of periodontal ligament tissue in experimental periodontal defects in dogs to the extent of 4/5, while GTR procedures result with the regeneration of only a half of the total defect length at 12 weeks post operatively. The most coronal part of the measured area was infiltrated by migrating epithelial cells which formed the junctional epithelium around the cemento-enamel junction. The mean length of the epithelial migration varied between 0.45 mm (˜8%) to 0.6 mm (˜11%) in all of the groups, with no statistically significant difference observed between different scaffolds and controls at both time points. The differences between 4^{th} and 12^{th} week measurements were also statistically insignificant.

The percent of newly formed bone (% NB) at 4^{th} week was highest in OIA-CGS with %34.4±6.88, followed by CI-CGS with %28.4±8.98 and FIA-CGS with %23.6±9.07. The level in control group remained at %11.2±3.7, thus up to 3-fold difference was observed with OIA-CGS compared to control. At 12^{th} week the % NB levels for the groups were slightly higher than the % NPDL(f) levels at that time point, with the highest percent in OIA-CGS (%88.8±5.63) and lowest one in control (%55.2±7.98) groups. The formation of new cementum on 5.5 mm-long (aRP-CEJ) root surface was also measured and the corresponding percent of NC at Week 4 was highest in CI-CGS with %17.2±7.33, followed by OIA-CGS (%14.6±7.64) and FIA-CGS (%12±6.24), and lowest in control with %7.6±3.36. However, the differences between the groups were not statistically significant. At 12^{th} week the newly formed cementum increased to levels approximately 5-fold of the 4^{th} week levels in cell-guiding scaffolds, with similar percent in CI-CGS (%77±6.4) and OIA-CGS (%75.6±9.02) and slightly lower in FIA-CGS (%67.4±8.29). Control levels (%30.4±6.73) were also 4-fold higher compared to 4^{th} week but the difference was more than 2-fold between cell-guiding scaffold groups and the control. The results indicate the effectiveness of cell-guiding scaffolds of the present invention in augmenting the regeneration of periodontal tissues, where new periodontal ligament and alveolar bone regeneration is ˜1.5-fold and new cementum formation is ˜2-fold greater than the levels achieved by the state-of-the art guided tissue regeneration technique utilizing barrier membranes allowing resident periodontal ligament regenerative cells to spontaneously regenerate the lost tissues in periodontal defects.

1. A cell-guiding scaffold composed of biodegradable and biocompatible natural biopolymers, synthetic polymers and/or their combination, incorporating growth and differentiation factors as chemoattractants or growth hormone, with interconnected pores and channels-containing defined microarchitecture, for induction of the regenerative cell migration, adhesion, proliferation and differentiation from the healthy tissues surrounding the periodontal defects, thereby facilitating the functional periodontal tissue regeneration.

2. A cell-guiding scaffold of claim 1, wherein said biocompatible and biodegradable natural biopolymers are selected from the group consisting of fibrinogen, fibrin, hyaluronic acid, collagen type I, collagen type III, fibronectin, laminin, vitronectin, gelatin, elastin, alginate and silk fibroin.

3. A cell-guiding scaffold of claim 1, wherein said synthetic polymers are selected from the group of polymers consisting of poly(lactic-co-glycolic acid), poly(lactic acid), poly(glycolic acid), poly(vinyl alcohol), and poly(ε-caprolacton).

4. A method for the fabrication of the cell-guiding scaffold of claim 1, wherein one or more of the natural biopolymers selected from the group consisting of fibrin, fibrinogen, laminin, fibronectin, collagen type I and collagen type III are combined with one or more of the synthetic polymers selected from the group consisting of poly(lactic-co-glycolic acid), poly(lactic acid), poly(glycolic acid), poly(vinyl alcohol), and poly(ε-caprolacton).

5. A method for the fabrication of the cell-guiding scaffold of claim 1, wherein one or more of the extracellular matrix proteins selected from the group consisting of laminin, fibronectin, vitronectin and collagen type I are used on the scaffold biomaterial surfaces as cell attachment enhancing substances.

6. A method for the fabrication of the cell-guiding scaffold of claim 5, wherein said extracellular matrix proteins selected from the group consisting of laminin, fibronectin, vitronectin and collagen type I are incorporated into the said scaffolds in a concentration ranging between 5-100 μg/ml, and more preferably between 10-50 μg/ml.

7. A method for the fabrication of the cell-guiding scaffold of claim 1, wherein one or more of the inorganic substances selected from the group consisting of calcium carbonate, calcium phosphate, hydroxyapatite and nanohydroxyapatite crystals are incorporated into the scaffold structure as osteoconductive agents.

8. A method for the fabrication of the cell-guiding scaffold of claim 1, wherein said growth and differentiation factors selected from the group consisting of basic fibroblast growth factor (bFGF), acidic fibroblast growth factor (aFGF), insulin-like growth factor-I (IGF-1), insulin-like growth factor-II (IGF-II), platelet-derived growth factor αβ (PDGF αβ), platelet-derived growth factor ββ (PDGF ββ), brain-derived neurotrophic factor (BDNF), transforming growth factor-β (TGF-β), bone morphogenetic protein-2 (BMP-2), bone morphogenetic protein-4 (BMP-4), bone morphogenetic protein-7 (BMP-7), and vascular endothelial growth factor (VEGF) are incorporated into the scaffold in different combinations and concentrations as chemoattractants, and proliferation and differentiation-inducing factors.

9. A method for the fabrication of the cell-guiding scaffold of claim 8, wherein said growth and differentiation factors are incorporated into the said scaffolds in a concentration ranging between 5-1000 μg/ml, and more preferably between 10-500 μg/ml, and still more preferably between 20-200 μg/ml.

10. A cell-guiding scaffold of claim 1, wherein said growth and differentiation factors of claim 8 are recombinant growth factors.

11. A method for the fabrication of the cell-guiding scaffold of claim 1, wherein said growth and differentiation factors are incorporated to the said natural biopolymers and/or synthetic polymers by the binding to heparin.

12. A cell-guiding scaffold of claim 1, wherein growth hormone (GH, somatotropin) is incorporated as cell proliferation inducer.

13. A cell-guiding scaffold of claim 1, incorporating one or more of the enamel matrix proteins selected from the group consisting of amelogenin, ameloblastin, enamelin, amelotin, odontogenic ameloblast associated protein (ODAM), and one of the dentin matrix proteins dentin derived phosphosphorin (DPP) and dentin sialoprotein (DSP) as cell differentiation inducers.

14. A cell-guiding scaffold of claim 1, wherein said microarchitecture contains predefined interconnected porous component and channels component in a structure conductive of osteoblastic, fibroblastic, angiogenic and cementoblastic regenerative cells' migration, proliferation and functional extracellular matrix synthesis and deposition.

15. A method for attaining the cell-guiding scaffold microarchitecture of claim 14, by using one or more of the fabrication techniques selected from the group of solvent-casting and porogen-leaching, phase separation and freeze-drying (lyophilization), rapid prototyping, and computer assisted solid free-form fabrication.

16. A cell-guiding scaffold of claim 14, wherein said predefined microarchitecture and regenerative cells result in a combination that facilitate the development of cementogenesis, osteogenesis, angiogenesis and fibrous connective tissue regeneration when the said scaffold is implanted in periodontal defect sites in mammals.

17. A method for attaining the cell-guiding scaffold microarchitecture of claim 14, wherein said porous component is composed of interconnected pores with diameter ranging between 10-500 μm, and more preferably between 50-250 μm.

18. A method for attaining the cell-guiding scaffold microarchitecture of claim 14, wherein said porous component is localized on the surface layer of the said scaffold that will face the bone tissue surface when placed in a periodontal defect site of a mammal.

19. A method for attaining the cell-guiding scaffold microarchitecture of claim 14, wherein said channels component is composed of interconnected channels with diameter ranging between 10-500 μm, and more preferably between 50-250 μm, and still more preferably between 100-200 μm.

20. A method for attaining the cell-guiding scaffold microarchitecture of claim 14, wherein said channels component is composed of channels with longitudinal, oblique and transverse orientation in different parts of the said scaffold structure.

21. A method for the surface modification of the cell-guiding scaffold of claim 1 with fibrin glue prior to the application to the prepared tooth root surfaces during periodontal surgical procedures in humans.

22. A cell-guiding scaffold of claim 1, wherein the said scaffold can be seeded with one or more of the regenerative cells selected from the group consisting of autologous periodontal ligament stem cells, cementoblastic cells, osteoblastic cells, osteoprogenitor cells, bone marrow-derived mesenchymal stem cells, adipose tissue-derived stem cells, dental follicle stem cells and genetic engineered cells in vitro prior to the application in periodontal regenerative procedures in humans.
